(19) United States
(12) Patent Application Publication
(10) Pub. No.: US 2024/0290489 A1
(43) Pub. Date: Aug. 29, 2024

(54) DIAGNOSIS AND MONITORING OF BRAIN CANCER

(71) Applicants: Cambridge Enterprise Limited, Cambridge, Cambridgeshire (GB); Stichting Amsterdam UMC, Amsterdam (NL)

(72) Inventors: Kevin Brindle, Cambridge, Cambridgeshire (GB); Richard Mair, Cambridge, Cambridgeshire (GB); Florent Moulière, Manchester (GB); Nitzan Rosenfeld, Cambridge, Cambridgeshire (GB); Christopher G. Smith, Cambridge, Cambridgeshire (GB)

(73) Assignees: Cambridge Enterprise Limited, Cambridge, Cambridgeshire (GB); Stichting Amsterdam UMC, Amsterdam (NL)

(21) Appl. No.: 18/573,418

(22) PCT Filed: Jul. 8, 2022
(86) PCT No.: PCT/EP2022/069203

§ 371 Date: Dec. 21, 2023

(30) Foreign Application Priority Data
Jul. 9, 2021 (GB) 2109941.1

(51) Int. Cl. G16H 50/20; G16B 20/20; G16B 40/20

(52) U.S. Cl. CPC G16H 50/20; G16B 20/20; G16B 40/20

(57) ABSTRACT

The present invention provides a computer-implemented method for analysing a urine sample from a subject. The method comprises providing the value of one or more cell-free DNA fragment size metrics for said sample, and determining whether the sample has a high or low likelihood of being from a brain cancer patient by providing said values of said cell-free DNA fragment size metrics as input to a machine learning model. The machine learning model is trained to classify sample data into one of at least two classes, the at least two classes comprising a first class having a high likelihood of being from a brain cancer patient and a second class having a low likelihood of being from a brain cancer patient. Methods for diagnosing or screening for brain cancer, detecting recurrence or residual disease, providing a prognosis or selecting a treatment for brain cancer are also described.

### FIELD OF THE INVENTION

The present invention relates in part to methods for diagnosing, treating and monitoring brain cancer by analysing urine samples. In particular, the methods of the invention find use in the diagnosis, treatment and monitoring of brain cancers such as glioma.

### BACKGROUND TO THE INVENTION

Primary brain tumours, which are diagnosed in over 260,000 patients worldwide annually (Wesseling & Capper, 2018), have a poor prognosis and lack effective treatments. Better methods for early detection and identification of tumour recurrence may enable the development of novel treatment strategies. The development of new treatments would also benefit from minimally invasive methods that characterise the evolving glioma genome (Westphal & Lamszus, 2015; Brennan et al, 2013). DNA analysis in liquid biopsies has the potential to replace or supplement current imaging-based monitoring techniques, which have limited effectiveness, and to provide the genomic information required for precision medicine whilst reducing the morbidity associated with repeated biopsy (Westphal & Lamszus, 2015; Kros et al, 2015; Mouliere et al, 2014). However, cell-free tumour DNA (ctDNA) is extremely challenging to detect in the plasma of patients with brain tumours as its fractional concentration (mutant allele fractions, MAF) is low and appears to be in the same range as that observed in plasma of patients with early stage carcinomas (Bettegowda et al, 2014; Zill et al, 2018). Reported detection rates for ctDNA in plasma of glioma patients are typically around 15%-30% (Bettegowda et al, 2014). Although higher rates of detection have been reported, the high frequency of alterations resulting from clonal hematopoiesis may confound these results (Zill et al, 2018; Piccioni et al, 2019; Pan et al, 2019). In addition to plasma, ctDNA has been detected in urine for some cancer types, however this has been limited largely to urothelial cancers, or patients with advanced cancers and high plasma tumour fraction (Patel et al, 2017; Dudley et al, 2019; Husain et al, 2017; Bosschieter et al, 2018; Hentschel et al, 2020). Cerebrospinal fluid (CSF) has been proposed as an alternative medium for brain tumour ctDNA analysis (De Mattos-Arruda et al, 2015; Wang et al, 2015; Mouliere et al, 2018b; Pentsova et al, 2016; Seoane et al, 2019; Pan et al, 2019, 2015), however detection sensitivity has remained poor in previous analyses (CSF detected in 42/85 patients, 49.4%) (Miller et al, 2019). In addition, CSF sampling via lumbar puncture is an invasive and painful procedure for patients and requires skilled medical staff, which severely limits its use for research, diagnosis and repeat sampling (Hasbun et al, 2001; Engelborghs et al, 2017).

Thus, compared to other disease types, detection of circulating cell-free tumour DNA (ctDNA) in patients with brain tumours, in particular gliomas (GBM), is challenging. Because CSF is both difficult to collect and associated with significant discomfort for the patient, it is unlikely that analysis of ctDNA in CSF will be considered as a viable approach for longitudinal sampling going forward. On the other hand, minimally invasive liquid biopsy, in the form of plasma or urine, don't face these same challenges, but their use is hampered by the presence of only minute levels of glioma-derived cfDNA signal.

Thus, there remains a need for approaches that can effectively detect ctDNA in patients with brain cancer, that do not suffer from the disadvantages of existing methods.

### BRIEF DESCRIPTION OF THE INVENTION

The present inventors have previously demonstrated that tumour cfDNA could be detected in plasma samples for a variety of cancers using a machine learning approach combining cfDNA fragmentation pattern information and somatic alteration analysis (Mouliere et al., 2018a). In particular, in Mouliere et al. (2018a), a random forest model including as predictive features (a) the proportion of fragments in the size ranges 160-180, 180-220 and 250-320, (b) the amplitude of oscillations in fragment size density with 10-bp (base pairs) periodicity, and (c) a feature quantifying the deviation from copy number neutrality (t-MAD, trimmed median absolute deviation from copy number neutrality) was found to have best performance in discriminating between healthy and cancer patients using plasma samples, when assessed on a cohort of samples from cancer types with low ctDNA in plasma (renal cancer, glioblastoma, bladder cancer, pancreatic cancer). This was also the subject of patent application WO 2020/094775, which is incorporated herein by reference. The present inventors hypothesised that differences in fragment lengths of circulating DNA could be present in urine samples as well. The present inventors further hypothesized that an approach specifically designed for detection of ctDNA in urine samples could be exploited to enhance sensitivity for detecting the presence of ctDNA for non-invasive genomic analysis of brain cancers. As explained above, this is a particularly challenging task even in fluids such as CSF, let alone in urine. As described in detail herein, the present inventors used a sequencing approach that preserves the structural properties of ctDNA, allowing them to determine the size profile of mutant ctDNA in matched CSF, plasma and urine samples from glioma patients. This demonstrated a shift towards shorter fragment sizes for mutant (tumour-derived) cfDNA in comparison to non-mutant cfDNA in CSF, plasma and urine samples, with different respective characteristics in each of the fluids. Based on this, they designed an approach specifically tailored to detect ctDNA in urine of brain cancer patients. Analysing urine fragmentation in samples from 5 patients with low grade glioma (LGG) and with high grade glioma (HGG), and 53 individuals without glioma, the inventors demonstrated that urine samples from glioma patients could be identified by analysing specific fragmentation patterns from shallow whole genome sequencing (sWGS, <1× coverage) data using machine learning classifiers. They discovered in particular that in this context the proportion of fragments in lower size ranges than those used in plasma were particularly informative, and that including features that capture these size ranges specifically as informative features for the classification improved the sensitivity and specificity of classification in the context of detecting ctDNA from brain tumours in urine samples.

Accordingly, in a first aspect the present invention provides a method for analysing a urine sample from a subject, the method comprising: providing the value of one or more cell-free DNA fragment size metrics for said sample; and determining whether the sample has a high or low likelihood of being from a brain cancer patient by providing said values of said cell-free DNA fragment size metrics as input to a machine learning model trained to classify sample data into one of at least two classes, the at least two classes comprising a first class having a high likelihood of being from a brain cancer patient and a second class having a low likelihood of being from a brain cancer patient, wherein the one or more cell-free DNA fragment size metrics comprise at least one metric representing the proportion of fragments in a size range that does not extend above 100 bp and that is between 10 and 100 bp wide.

The present inventors have discovered that the cfDNA fragmentation profile in urine samples could be used to discriminate between samples that are likely to contain ctDNA from brain cancer and samples that are unlikely to contain ctDNA from brain cancer, and that such a discrimination was particularly improved by investigating the range of sizes below 100 in more detail than was previously done for plasma samples. This is based at least in part on the discovery that cfDNA fragmentation patterns are different in urine and plasma samples, and further that samples from patients with other central nervous diseases also show fragmentation patterns that differ from those seen in samples from healthy patients, such that an approach specifically tailored to the particular size distribution features in these types of patients enhances the ability to discriminate between patients with and without brain malignancies.

All of the methods described herein may be computer implemented unless context indicates otherwise. As the skilled person understands, the complexity of the operations described herein (due at least to the complexity of analysing sequencing data, training a machine learning model, obtaining a distribution of fragment size from sequencing data etc. as described herein, particularly in view of the amount of data that is typically generated by DNA sequencing) are such that they are beyond the reach of a mental activity. Thus, unless context indicates otherwise (e.g. where sample preparation or acquisition steps are described), all steps of the methods described herein are computer implemented.

The one or more cell-free DNA fragment size metrics may comprise a plurality of metrics representing the proportion of fragments in respective size ranges. The respective size ranges may be substantially non-overlapping. Two size ranges may be substantially non-overlapping when the proportion of the size ranges that is common between them is smaller than the proportion of each size range that is unique to itself. For example, size ranges that overlap by a common range that represents less than 10% of each of the respective size ranges (where the exact percentage may be different for the respective size range depending on their size) may be considered to be substantially non-overlapping. The one or more cell-free DNA fragment size metrics may comprise a plurality of metrics representing the proportion of fragments in respective size ranges that are each between 0 and 300 bp. Each of the respective size ranges may be between 10 and 100 bp wide. The one or more cell-free DNA fragment size metrics may comprise a metric representing the amplitude of oscillations in fragment size density with approximately 10 bp periodicity in a particular size range. The particular size range may be between approximately 50 bp and approximately 140 bp.

The one or more cell-free DNA fragment size metrics may comprise a plurality of metrics representing the proportion of fragments in respective substantially non-overlapping size ranges between 0 and 150 bp. The one or more cell-free DNA fragment size metrics may comprise at least 2 or at least 3 metrics representing the proportion of fragments in respective substantially non-overlapping size ranges between 0 and 150 bp. The size range or each of the respective size ranges may be between 20 and 100 bp wide, between 20 and 80 bp wide, between 20 and 50 bp wide, at least 10 bp wide, at least 20 bp wide, at least 30 bp wide, at most 100 bp wide, at most 90 bp wide, at most 80 bp wide, at most 70 bp wide, at most 60 bp wide, at most 50 bp wide, about 20 bp wide, about 30 bp wide, about 40 bp wide or about 50 bp wide. The one or more cell-free DNA fragment size metrics may comprise one or more metrics representing the proportion of fragments in the 30-90 bp range and/or one or more metrics representing the proportion of fragments in the 90-150 bp range. The one or more metric representing the proportion of fragments in the 30-90 bp range may comprise a metric representing the proportion of fragments in the 30-60 bp range and/or a metric representing the proportion of fragments in the 60-90 bp range. The one or more metric representing the proportion of fragments in the 90-150 bp range may comprise a metric representing the proportion of fragments in the 90-120 bp range and/or a metric representing the proportion of fragments in the 120-150 bp range. The one or more cell-free DNA fragment size metrics may comprise a metric representing the proportion of fragments in a plurality of ranges selected from the following ranges: 30-60 bp, 60-90 bp, 90-120 bp, 120-150, 150-180, 180-210, 240-270 and 270-300. The cell-free DNA fragment size metrics may further comprise a metric representing the amplitude of oscillations in fragment size density with 10 bp periodicity in a particular size range. The cell-free DNA fragment size metrics may further comprise a metric representing the proportion of fragments in each of the following ranges: 30-60 bp, 60-90 bp, 90-120 bp, 120-150, 150-180, 180-210, 240-270 and 270-300. As the skilled person understands, the reference to e.g. the 60-90 size range may encompass a range that starts at 61, for example when a 30-60 size range is also used in order to avoid double counting. In other words, strictly non-overlapping equivalents of each of the combinations of ranges described are also envisaged.

Providing the value of one or more cell-free DNA fragment size metrics for said sample may comprise: providing data representing fragment sizes of cell-free DNA fragments obtained from said sample; and determining the value of the one or more cell-free DNA fragment size metrics from the data representing fragment sizes of cell-free DNA fragments obtained from said sample. The step of providing data representing fragment sizes of cell-free DNA fragments obtained from said sample may comprise sequencing DNA from said sample and/or obtaining a urine sample from said subject and/or processing a urine sample from said subject or a sample of DNA derived therefrom. The data representing fragment sizes of the cell-free DNA fragments may comprise fragment sizes inferred from sequence data (e.g. sequence reads), fragment sizes determined by fluorimetry, or fragment sizes determined by densitometry. Alternatively, the data representing fragment sizes of cell-free DNA fragments obtained from the sample may comprise sequence data. The step of providing data representing fragment sizes of cell-free DNA fragments may comprise determining the lengths of cfDNA fragments from sequence data and/or determining the distribution of lengths of cfDNA fragments from sequence data. The sequence data may have been obtained using paired-end sequencing. The sequence data may have been obtained using a ligation-based approach do obtain a sequencing library. The sequencing library may be an indexed sequencing library. The present inventors have found the user of paired-end sequencing and/or a ligation-based strategy for library preparation to result in particularly higher recovery rates of cfDNA. This may in turn further improve the performance of the methods described herein. The step of providing data (e.g. sequence data, data representing fragment sizes of cell-free DNA fragments, the value of one or more cell-free DNA fragment size metrics for said sample) for a sample from the subject may comprise or consist of receiving data from a user (for example through a user interface), from one or more computing device (s), or from one or more data stores or databases.

The step of providing data representing fragment sizes of cell-free DNA fragments obtained from said sample may further comprise sequencing (or otherwise determining the sequence composition of genomic material present in a sample) one or more samples from the subject, wherein the one or more samples is/are urine samples from the subject, cfDNA-containing samples derived from urine samples from the subject, or samples derived therefrom such as e.g. by purification (including e.g. size selection to remove very large fragments such as e.g. genomic DNA fragments), extraction, library preparation, etc. Size selection may comprise an in vitro size selection that is performed on DNA extracted from a urine sample and/or is performed on a library created from DNA extracted from a urine sample. For example, in vitro size selection may comprises agarose gel electrophoresis or bead-based size selection. Instead or in addition to in vitro size selection, size selection may comprise an in silico size selection that is performed on sequence reads. The value of one or more cell-free DNA fragment size metrics for said sample may be derived from sequence data. In convenient embodiments, the sequence data may be whole genome sequencing (WGS) data, paired-end sequencing data, hybrid-capture sequencing and/or shallow whole genome sequencing (sWGS) data. In general, it is believed that the methods described herein would provide useful results using any type of data from which cell-free DNA fragment size information can be obtained. This includes for example sequencing data, fluorimetry data and densitometry data. Sequencing data is believed to be a particularly convenient type of data (at least because it is generally available). particularly when the sequencing includes a step of ligation and paired-end sequencing (as this can result in high cfDNA recovery rates). The sequencing data may be whole genome (such as e.g. WGS), or may use a capture-based approach (such as e.g. hybrid-capture sequencing). sWGS data may refer to WGS data that has <0.4× depth of coverage. The present inventors have discovered that sWGS was able to provide enough information to analyse urine samples as described herein, thereby providing a cost-effective way of diagnosing brain cancer in a non-invasive manner, increasing the scope of clinical applicability of the methods described.

The method may further comprise obtaining, from the subject, one or more urine samples. The method may further comprise processing a urine sample obtained from the subject or a DNA sample derived therefrom, for example by purification, extraction, library preparation, etc. The method may further comprise providing to a user, for example through a user interface, an output of the method such as a determination of whether the sample has a high or low likelihood of being from a brain cancer patient, a probabilistic score provided by the machine learning model and/or a value derived therefrom or associated therewith.

The machine learning model may have been trained using training data comprising the values of cfDNA size metrics for a plurality of urine samples from subjects with brain cancer and for a plurality of urine samples from subjects that do not have brain cancer. The subjects that do not have brain cancer comprise healthy subjects and subjects with non-malignant central nervous system diseases. For example, data from patients that have non-malignant central nervous system diseases selected from the following set may be used: cervical myelopathy, cerebral artery aneurysm, hydrocephalus and Parkinson's disease. The machine learning model may be a random forest model, a logistic regression model, a support vector machine, or a generalised linear model. A generalised linear model may be a regularized generalised linear model. The machine learning model may provide an output that is a probabilistic score, such as a probability of belonging to the high likelihood class or a probability of correct classification, e.g., a probability that the sample in question has been classified correctly. The machine learning may provide an output that is a probabilistic score, and determining whether the sample has a high or low likelihood of being from a brain cancer patient may comprise comparing the probabilistic score to a threshold, for example a threshold determined based on the training data as one that most accurately classifies training samples on the high/low likelihood category. The performance of the machine learning model when trained on the training set may be assessed by the area under the curve (AUC) value from a receiver operating characteristic (ROC) analysis. Generally a model showing the highest AUC value may be selected as having the best performance. The machine learning model may have been trained on a training set comprising at least 10, 20, 30, 40 or at least 50 samples from subjects that do not have brain cancer and at least 10, 20, 30, or at least 40 samples from subjects known to have a brain cancer.

The urine sample may be from a subject having or suspected of having a brain cancer. The brain cancer may be a glioma, a meningioma, a pituitary adenoma, a glioblastoma, a medulloblastoma, an oligodendroglioma, a brain metastasis. The brain cancer may be a glioma. The subject may be a human. A glioma may be a high grade glioma or a low grade glioma. A brain metastasis may be a metastasis located in the brain, associated with a cancer of any origin. The method may be a method for detecting the presence of, growth of, prognosis of, regression of, treatment response of, residual disease or recurrence of a brain cancer in a subject from which the sample has been obtained. The urine sample may have been obtained prior to the subject having undergone treatment with a cancer therapy. The urine sample may have been obtained subsequent to the subject having undergone treatment with a cancer therapy. The method may be carried out on a sample obtained prior to a cancer treatment of the subject and on a sample obtained following the cancer treatment of the subject. The urine sample may be or have been processed within 12 hours, within 4 hours, within 2 hours or within an hour of collection. The processing may comprise refrigeration, freezing, centrifugation, and/or mixing with one or more preserving compounds such as EDTA. The sample may have been obtained from the subject in a primary care setting, in a hospital, or at any other location such as e.g. privately by the subject (e.g. at home). In particular, the sample may have been obtained at a location that is different from the location at which the sample is processed (e.g. to preserve it, extract DNA, derive a library, sequence the DNA in the sample, etc.) and/or the location at which the sequence data is analysed to provide the value of one or more cell-free DNA fragment size metrics for said sample and/or the location at which said values are analysed as described herein. In particular, each of the above may be performed at different locations. Further, any data analysis step may be performed over a distributed network such as e.g. on the cloud. Further, each of the above may be performed at locations that are not primary care locations or hospitals. Indeed, it is an advantage of the invention that an analysis can be performed without requiring trained medical staff, contrary to diagnosis/monitoring methods that require an invasive step (such as e.g. collection of blood or csf) or specialised medical equipment (such as e.g. medical imaging).

In a second aspect the present invention provides a method for analysing a urine sample from a subject, comprising: analysing a urine sample, a DNA sample derived from a urine sample, or a library derived from a urine sample, wherein the sample has been obtained from the subject, to determine fragment sizes of nucleic acid fragments in said sample or said library; and carrying out the method of the first aspect of the invention using the fragment sizes. Also described is a method for analysing a urine sample from a subject, comprising: sequencing a DNA sample derived from the urine sample, or a library derived from the urine sample, that has been obtained from the subject to obtain a plurality of sequence reads; processing the sequence reads to determine data representing fragment sizes of cfDNA fragments obtained from said sample; and carrying out the method of the first aspect of the invention using the data. Processing the sequence reads may comprise one or more of the following steps: aligning sequence reads to a reference genome of the same species as the subject (e.g. the human reference genome GRCh37 for a human subject); removal of contaminating adapter sequences; removal of PCR and optical duplicates; removal of sequence reads of low mapping quality; and if multiplex sequencing, de-multiplexing by excluding mismatches in sequencing barcodes.

In accordance with any aspect of the invention, the fragment sizes of cfDNA fragments may be inferred from sequence reads using the mapping locations of the read ends in the genome following alignment of the sequence reads with the reference genome of the species from which the sample was obtained. In accordance with any aspect of the present invention the sample may be or may have been subjected to one or more processing steps to remove whole cells, for example by centrifugation. In particular cases the sequence reads may comprise paired-end reads generated by sequencing DNA from both ends of the fragments present in a library generated from the urine sample or DNA sample derived therefrom. The original length of the DNA fragments in the cfDNA containing sample may be inferred using the mapping locations of the read ends in the genome following alignment of the sequence reads with the reference genome of the species from which the sample was obtained (e.g. the human reference genome GRCh37 for a human subject). In accordance with any aspect of the present invention, the subject may be mammalian, a human, a companion animal (e.g. a dog or cat), a laboratory animal (e.g. a mouse, rat, rabbit, pig or non-human primate), a domestic or farm animal (e.g. a pig, cow, horse or sheep). Preferably, the subject is a human patient. In some cases, the subject is a human patient who has been diagnosed with, is suspected of having or has been classified as at risk of developing, a brain cancer.

According to a third aspect, there is provided a method of diagnosing a subject suspected of having a brain cancer as likely to have brain cancer, the method comprising: analysing one or more urine samples from the subject using the method of any embodiment of the first aspect to determine whether the one or more samples have a high or low likelihood of being from a brain cancer patient; and diagnosing the subject as likely to have a brain cancer if one or more of the one or more urine samples are determined to have a high likelihood of being from a brain cancer patient. A subject suspected of having a brain cancer may be a subject belonging to a population considered to be at risk of developing brain cancer. The risk may be low, and may be based on e.g. age, medical history, family history, the presence of genetic markers of risk in the subject or their family, etc. Thus, the method may be used for screening of a population of subjects. As such, also described herein is a method of screening for brain cancer in a population of subjects, the method comprising: analysing one or more urine samples from the subjects using the method of any embodiment of the first aspect to determine whether the one or more samples have a high or low likelihood of being from a brain cancer patient; and diagnosing a subject as likely to have a brain cancer if one or more of the one or more urine samples from the subject are determined to have a high likelihood of being from a brain cancer patient.

According to a fourth aspect, there is provided a method of selecting a subject suspected of having a brain cancer for treatment with a cancer therapy, the method comprising characterising a urine sample obtained from the subject as having a high or low likelihood of being from a cancer patient using the method of any embodiment of the first aspect, and selecting the subject for treatment with the cancer therapy if the sample is characterised as having a high likelihood of being from a brain cancer patient. The subject may have been previously treated for brain cancer, and the brain cancer therapy may be a therapy that has been previously used for the subject or a different therapy. For example, the cancer therapy may be a cancer therapy that has not previously been used for the subject. The method may further comprise obtaining an image-based analysis for the subject such as e.g. a brain MRI. In such embodiments, the step of selecting the subject for treatment with the cancer therapy may depend on the result of the image-based analysis as well as the analysis of the urine sample. For example, a different course of treatment may be selected if the sample is characterised as having a high likelihood of being from a brain cancer patient, depending on the result of the image-based diagnosis.

According to a fifth aspect, there is provided method of selecting a subject suspected of having a brain cancer for further diagnostic test, the method comprising characterising a urine sample obtained from the subject as having a high or low likelihood of being from a cancer patient using the method of any embodiment of the first aspect, and selecting the subject for further diagnostic test if the sample is characterised as having a high likelihood of being from a brain cancer patient. The further diagnostic test may be an invasive diagnostic test and/or an imaging-based test. An invasive diagnostic test may comprise a biopsy, such as e.g. a blood, CSF or tissue biopsy. An imaging-based test may comprise a brain MRI.

According to a sixth aspect, there is provided a method of detecting recurrence of a brain cancer in a subject, the method comprising characterising a urine sample obtained from the subject as having a high or low likelihood of being from a cancer patient using the method of any embodiment of the first aspect, and determining that recurrence is likely to have occurred if the sample is characterised as having a high likelihood of being from a brain cancer patient. According to a related aspect, there is provided a method of detecting residual disease in a subject with brain cancer, the method comprising characterising a urine sample obtained from the subject as having a high or low likelihood of being from a cancer patient using the method of any embodiment of the first aspect, and determining that residual disease is likely to be present if the sample is characterised as having a high likelihood of being from a brain cancer patient. In accordance with any aspect described herein, the subject may have been previously treated for brain cancer. The methods according to any embodiment of any aspect may be repeated using urine samples that have been obtained from the subject at a plurality of times. For example, this may be performed in order to monitor the presence or absence of recurrence of a brain cancer in the subject, or to diagnose a brain cancer in a subject (e.g. a subject at risk of developing brain cancer). One of the advantages of the invention over previous methods to diagnose initial/recurrent brain cancer is that the method is non-invasive and simple to implement, thereby expanding the possibilities in terms of frequency of monitoring. For example, the method may be repeated using urine samples that have been obtained from the subject monthly, weekly or even daily. As a result, the sensitivity of detection of a brain cancer or recurrence thereof may be increased, thereby improving the chances of a good prognosis for the subject as the cancer can be treated earlier than would have otherwise been possible. This may be particularly advantageous in the context of detecting recurrence in a subject previously treated for brain cancer.

According to a further aspect, there is provided a method of monitoring brain cancer in a subject previously treated for brain cancer, the method comprising characterising a urine sample obtained from the subject as having a high or low likelihood of being from a brain cancer patient using a method of any embodiment of the first aspect. The method may further comprise determining that the previous course of treatment was ineffective and/or that the subject's cancer has relapsed if the urine sample obtained from the subject is characterised as having a high likelihood of being from a brain cancer patient. The method may further comprise selecting the subject for treatment with a brain cancer therapy if the urine sample obtained from the subject is characterised as having a high likelihood of being from a brain cancer patient. According to a further aspect, there is provided a method of treating a brain cancer in a subject, the method comprising characterising a urine sample obtained from the subject as having a high or low likelihood of being from a cancer patient using the method of any embodiment of the first aspect, and treating the subject with a cancer therapy if the sample is characterised as having a high likelihood of being from a brain cancer patient.

According to a further aspect, there is provided a method of providing a prognosis for a subject who has been diagnosed with a brain cancer, the method comprising characterising a urine sample obtained from the subject as having a high or low likelihood of being from a brain cancer patient, wherein if the sample is characterised as having a high likelihood of being from a brain cancer patient, the subject is likely to have a poorer prognosis than a subject from which a urine sample is characterised as having a low likelihood of being from a brain cancer patient. The method may comprise providing said values of said cell-free DNA fragment size metrics as input to a machine learning model trained to classify sample data into one of a plurality of classes, the plurality of classes associated with different likelihoods of being from a brain cancer patient, wherein the plurality of classes are associated with different prognosis. For example, the plurality of classes may comprise a first class associated with a high likelihood of being from a brain cancer patient, a second class associated with a low likelihood of being from a brain cancer patient, and one or more further classes associated with intermediate likelihoods of being from a brain cancer patient, wherein subjects in the first class have poorer prognosis than subjects in the second and further classes, optionally wherein subjects in at least one of the further classes have poorer prognosis than subjects in the second class.

The methods of any aspect described herein may further comprise outputting a result of the method, for example through a user interface. The result may be selected from a classification of a sample in the high/low likelihood class, a probabilistic score indicating the likelihood of the sample being from a brain cancer patient, or information derived therefrom such as a prognosis, therapeutic or diagnosis indication. The method according to any aspect may comprise one or more of the following steps: subjecting the subject to one or more further diagnostic tests if the sample has been identified as likely to be from a brain cancer patient, optionally wherein the one or more further diagnostic tests are selected from an imaging based test, and a blood, plasma or CSF-based analysis; detecting the presence of one or more genetic alterations in the sequence data obtained from the urine sample; selecting the subject for treatment with a cancer therapy, and/or treating the subject with a cancer therapy; selecting the subject for further monitoring comprising repeating the method at a later time point.

According to a further aspect, there is provided a method for providing a tool for analysing a urine sample, the method comprising: providing the value of one or more cell-free DNA fragment size metrics for a plurality of training urine samples associated with known brain cancer status, wherein the one or more cell-free DNA fragment size metrics comprise at least one metric representing the proportion of fragments in a size range that does not extend above 100 bp and that is between 10 and 100 bp wide; and training a machine learning model to classify sample data into one of at least two classes, the at least two classes comprising a first class having a high likelihood of being from a brain cancer patient and a second class having a low likelihood of being from a brain cancer patient. The method of the present aspect may have any of the features described in relation to the first aspect. The machine learning model may be trained predict, based on said values of said one or more fragment size metrics, the likelihood of each sample being from a brain cancer patient, and to identify a threshold that applies to said likelihood and that classifies samples between at least two classes comprising a first class having a high likelihood of being from a brain cancer patient and a second class having a low likelihood of being from a brain cancer patient. The method may further comprise providing the trained machine learning model or one or more parameters thereof to a user, e.g. via a user interface, or to a computing device, or writing the trained machine learning model or more parameters thereof on a computer readable medium.

According to a further aspect, there is provided a system comprising: a processor; and a computer readable medium comprising instructions that, when executed by the processor, cause the processor to perform the (computer-implemented) steps of the method of any preceding aspect.

According to a further aspect, there is provided a non-transitory computer readable medium or media comprising instructions that, when executed by at least one processor, cause the at least one processor to perform the method of any embodiment of any aspect described herein.

According to a further aspect, there is provided a computer program comprising code which, when the code is executed on a computer, causes the computer to perform the method of any embodiment of any aspect described herein.

Embodiments of the present invention will now be described by way of example and not limitation with reference to the accompanying figures. However various further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows flow diagrams illustrating, in schematic form, a method for analysing a urine sample according to the disclosure (A), and a method for providing a diagnosis, prognosis or treatment recommendation according to the disclosure (B).

FIG. 2 shows an embodiment of a system for analysing a urine sample and/or for providing a diagnosis, prognosis or treatment recommendation according to the disclosure.

FIG. 3 shows fragment size distributions for mutant (blue) and non-mutant (red) cfDNA reads, determined from capture sequencing data for CSF samples (A), plasma samples (B) and urine samples (C). The data shows that mutant cfDNA has shorter fragments than non-mutant cfDNA in the CSF, plasma and urine samples of glioma patients.

FIG. 4 shows data investigating the influence of the age of the subject on the urine cfDNA fragmentation in healthy individuals. Colours represent individuals <35 years old (n=8, blue), between 35 and 45 years old (n=9, yellow), and >45 years old (n=8, grey). Age is unknown for 1 individual (not shown). The median age of the healthy individuals is 41 years old (range: 23-61). A. Median cfDNA size distribution. B. Median empirical cumulative distribution (ecdf). KS-test showed no significant difference. C. Proportion of cfDNA fragments <100 bp depending on the age group. No significant difference between the group can be detected (Wilcoxon-test).

FIG. 5 shows data indicating that cfDNA fragmentation patterns are altered in the urine of HGG and LGG patients when compared to healthy controls and other CNS diseases. A. Median size distribution of urine cfDNA fragments determined from paired-end sWGS (<1× coverage) of 26 healthy controls (in grey), 27 patients with other CNS diseases (cerebral aneurysm, and myeloneuropathy, in blue), 5 patients with LGG (in orange), and 30 HGG patients (35 samples, in red). Samples from LGG and HGG patients were collected at baseline. B. Median of the cumulative distribution function of the urine cfDNA fragment sizes of the patients included in this study. C. Proportion of fragment sizes between 30-60 bp in the urine of cfDNA from healthy controls (grey), other non-cancer CNS pathologies (light blue), LGG (orange), and HGG (red). Wilcoxson-test comparing the boxplots are added. Horizontal line within the bars represent median of the underlying population. Boxplot whiskers show 1.5 inter-quantile range of highest and lowest quartile.

FIG. 6 shows data demonstrating that cfDNA fragmentation patterns enable classification of glioma patients from controls. A. Schematic of the features extracted from the global cfDNA fragmentation patterns of urine samples. 10 features were calculated from the cfDNA fragments size (the proportion of fragments in specific size ranges: P30_60, P61_90, P91_120, P121_150, P151_180, P181_210, P211_240, P241_270, P271_300; and the amplitude of the 10 bp oscillations: OSC_10 bp). B. Workflow for the predictive analysis combining the urine fragment size features via LR, RF, SVM and GLMEN models. sWGS data from 40 urine samples from patients with gliomas and 53 urine samples from controls were split into 5 subsets for training/validation (80% of the samples) and testing (20% of the samples), according to a 5 fold cross-validation approach and 50 random iterations (see Methods). C. Principal component analysis comparing cancer (HGG and LGG) and control samples (healthy and other CNS diseases) using data from the urine fragmentation features. Red arrows indicate features tested during the predictive analysis. D. tSNE analysis comparing cancer and control samples using data from the same urine fragmentation features. E. ROC curves for binary classification of cancer and controls for each of the individual fragmentation features analysed. AUC values are added to the plots. F. AUC distribution for the unseen test-set (samples from patients with gliomas, 40; controls, 53) for four predictive models (LR, GLMEN, RF, SVM) trained and optimized following the scheme described in B and the Materials and Methods section. For each models are shown the AUC for the 50 iterations (i.e. each point is the AUC for one of the iterations). Horizontal line within the bars represent median of the underlying population. Boxplot whiskers show 1.5 inter-quantile range of highest and lowest quartile. G. Accuracy were compared for the 4 classifiers and 50 iterations on the unseen test-set of baseline and follow-up samples (19 samples). For each models are shown the AUC for the 50 iterations (i.e. each point is the AUC for one of the iterations). Horizontal line within the bars represent median of the underlying population. Boxplot whiskers show 1.5 inter-quantile range of highest and lowest quartile.

FIG. 7 shows the results of clustering of cfDNA fragmentation features recovered from sWGS using 10 bp binning. A. Principal component analysis comparing cancer (HGG and LGG) and control samples (healthy and other CNS diseases) using data from the urine fragmentation features. Red arrows indicate features tested during the predictive analysis. Fragmentation features were calculated from the cfDNA fragments size (The proportion of cfDNA fragments was calculated every 10 bp bins between 30 and 300 bp); and the amplitude of the 10 bp oscillations: OSC_10 bp). B. tSNE analysis comparing cancer and control samples using data from the same urine fragmentation features.

FIG. 8 shows the results of an evaluation of the fragmentation features determined from sWGS of urine samples using the 30 bp binning. A. Correlation matrix of the 10 fragmentation features determined by sWGS from the 74 urine samples included in the training and validation dataset of the classifier models. The correlation score was estimated for each cross-comparison, and the value displayed on as a color intensity (red=−1, blue=1), values indicated. B. Ranking of the individual features importance calculated with a Learning Vector Quantization (LVQ) model.

FIG. 9 shows correlation matrices for fragmentation features determined by sWGS from the 74 urine samples included in the training and validation dataset of the classifier models, using different sets of fragmentation features. A. 10 bp bins between 0 and 400 bp, and amplitude of the 10 bp oscillations: OSC_10 bp. B. 30 bp bins between 0 and 390 bp, OSC_10 bp. C. 50 bp bins between 0 and 400 bp, OSC_10 bp. D. 100 bp bins between 0 and 400 bp, OSC_10 bp.

FIG. 10 shows principal component analyses comparing cancer (HGG and LGG) and control samples (healthy and other CNS diseases) using data from the urine fragmentation features in FIG. 9. A. 10 bp bins between 0 and 400 bp, and amplitude of the 10 bp oscillations: OSC_10 bp. B. 30 bp bins between 0 and 390 bp, OSC_10 bp. C. 50 bp bins between 0 and 400 bp, OSC_10 bp. D. 100 bp bins between 0 and 400 bp, OSC_10 bp.

FIG. 11 shows the AUC distributions for the unseen test-set (samples from patients with gliomas, 40; controls, 53) for LR models using various sets of fragmentation features (from left to right: feature set in FIG. 9B excluding the P30-60 feature; P30-60 feature only; feature set in FIG. 9B excluding the P60-90 feature; P60-90 feature only; feature set in FIG. 9B excluding all features below 150 and including a P20-150 feature; feature set in FIG. 9A; feature set in FIG. 9D; feature set in FIG. 9C). For each models are shown the AUC for the 20 iterations (i.e. each point is the AUC for one of the iterations). Horizontal line within the bars represent median of the underlying population. Boxplot whiskers show 1.5 inter-quantile range of highest and lowest quartile.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

In describing the present invention, the following terms will be employed, and are intended to be defined as indicated below.
“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

A “sample” as used herein may be a biological sample, such as a cell-free DNA sample, a cell (including a circulating tumour cell) or tissue sample (e.g. a biopsy), a biological fluid, an extract (e.g. a protein or DNA extract obtained from the subject). Within the context of the present invention, the sample may be a urine sample, or a sample derived therefrom. The sample may be one which has been freshly obtained from the subject or may be one which has been processed and/or stored prior to making a determination (e.g. frozen, fixed or subjected to one or more purification, enrichment or extractions steps, including centrifugation). The sample may be derived from one or more of the above biological samples via a process of enrichment or amplification. For example, the sample may comprise a DNA library generated from the biological sample and may optionally be a barcoded or otherwise tagged DNA library. A plurality of samples may be taken from a single patient, e.g. serially during a course of treatment. Moreover, a plurality of samples may be taken from a plurality of patients. Sample preparation may be as described in the Materials and Methods section herein.

The term “sequence data” refers to information that is indicative of the presence and/or amount of genomic material in a sample that has a particular sequence. Such information may be obtained using sequencing technologies, such as e.g. next generation sequencing (NGS, such as e.g. whole exome sequencing (WES), whole genome sequencing (WGS), or sequencing of captured genomic loci (targeted or panel sequencing)), or using array technologies, such as e.g. SNP arrays, or other molecular counting assays. When NGS technologies are used, the sequence data may comprise a count of the number of sequencing reads (also referred to as “sequence reads” or “sequence read data”) that have a particular sequence. When non-digital technologies are used such as array technology, the sequence data may comprise a signal (e.g. an intensity value) that is indicative of the number of sequences in the sample that have a particular sequence, for example by comparison to an appropriate control. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular genomic location. Sequence reads data may be provided or obtained directly, e.g., by sequencing the cfDNA sample or library or by obtaining or being provided with sequencing data that has already been generated, for example by retrieving sequence read data from a non-volatile or volatile computer memory, data store or network location. Where the sequence reads are obtained by sequencing a sample, the median mass of input DNA may in some cases be in the range 1-100 ng, e.g., 2-50 ng or 3-10 ng. The DNA may be amplified to obtain a library having, e.g. 100-1000 ng of DNA. The library may be obtained using a ligation-based approach. The sequencing may be paired-end sequencing. The sequence reads may be in a suitable data format, such as FASTQ, SAM or BAM. The sequence read data, e.g., FASTQ files, may be subjected to one or more processing or clean-up steps prior to or as part of the step of reads collapsing into read families. For example, the sequence data files may be processed using one or more tools selected from as FastQC v0.11.5, a tool to remove adaptor sequences (e.g. cutadapt v1.9.1). The sequence reads (e.g. trimmed sequence reads) may be aligned to an appropriate reference genome (or may have been previously aligned to an appropriate reference sequence, e.g. in the case of SAM/BAM files), for example, the human reference genome GRCh37 for a human subject. As used herein “read” or “sequencing read” may be taken to mean the sequence that has been read from one molecule and read once. Each molecule can be read any number of times, depending on the sequencing performed.

The present invention relates broadly to the use of cfDNA fragment size metrics to characterise a urine sample from a subject. The term “cfDNA fragment size metric” refers to any metric that can be derived from a distribution of the size of cfDNA fragments in a sample. Within the context of the present invention, a cfDNA fragment size metric includes at least one metric indicative of the proportion of fragments within a particular size range. A size range may be expressed using numbers of base pairs (bp). For example, the size range 30-60 bp refers to the fragments that are between 30 bp and 60 bp in length. A metric indicative of the proportion of fragments within a size range may be a normalised number of fragments that have a length within said size range. The normalised number of fragment in a size range may be equal to the proportion of fragments in said range if the number of fragments is normalised using the total number of fragments in the sample or the total number of fragments within a predetermined size range that comprises the size range and optionally any other size range for which a metric may be calculated. A metric indicative of the proportion of fragments within a size range may be the value of a density function obtained from the distribution of fragments sizes in the sample. A cfDNA fragment size metric may be a metric that is obtained from the distribution of fragment sizes in the sample and that quantifies an aspect of the shape of the distribution, such as e.g. the amplitude of oscillations (optionally with a predetermined approximate periodicity such as e.g. 10 bp) within a predetermined range (e.g. 50-140 bp) of the distribution. Such a metric may be obtained by determining the height of local maxima and minima in the distribution for a sample within the predetermined range. Such a metric may be obtained by identifying local maxima and minima for each of a plurality of samples, within the predetermined range, estimating the average position of each maximum and minimum across the plurality of samples, and using the height of the distribution at each of these positions for a candidate sample to calculate the amplitude of oscillations for said candidate sample. An amplitude of oscillations may be obtained for a plurality of maxima and minima by summing the height of the maxima and subtracting the sum of the height of the minima. The height of a maximum/minimum may be defined as the number of fragments with the length corresponding to said maximum/minimum divided by the total number of fragments. Identifying local maxima/minima may comprise selecting positions y (i.e. sizes) such that the y is the largest value in the interval [y−2, y+2]. Any other method of identifying local minima/maxima in a distribution may be used. When the positions of maxima/minima are empirically defined (i.e. based on the distributions observed in one or more samples), the periodicity of the oscillation may not be exactly equal to a predetermined frequency. In particular, the distance between maxima or minima may not be exactly constant, and may vary slightly within the size range in which the periodic oscillations are observed. Thus, reference to periodic oscillations of e.g. 10 bp periodicity may in practice refer to peaks that are between e.g. 8 and 12 bp apart. A set of peak locations may be obtained from a plurality of training samples, for example samples from patients that have been identified as having cancer (e.g. brain cancer).

As used herein “treatment” refers to reducing, alleviating or eliminating one or more symptoms of the disease which is being treated, relative to the symptoms prior to treatment.

As used herein, the term “machine learning model” refers to a mathematical model that has been trained to predict one or more output values based on input data, where training refers to the process of learning, using training data, the parameters of the mathematical model that result in a model that can predict outputs values with minimal error compared to comparative (known) values associated with the training data (where these comparative values are commonly referred to as “labels”). The term “machine learning algorithm” or “machine learning method” refers to an algorithm or method that trains and/or deploys a machine learning model. “Classifier” or “classification algorithm” may be a machine learning model or algorithm that maps input data, such as a cfDNA fragment size features, to a category, such as cancerous or non-cancerous origin. A classifier may produce as output a probabilistic score, which reflects the likelihood that an observation belongs to particular category, In some embodiments, the present invention provides methods for detecting, classifying, prognosticating, or monitoring cancer in subjects. In particular, data obtained from sequence analysis, such as fragment length may be evaluated using one or more classification algorithms. The machine learning approaches used herein may be termed “supervised” as a training set of samples with known class or outcome is used to produce a mathematical model which is then evaluated with independent validation data sets. Here, a “training set” of sequence information, e.g. fragmentation features, is used to construct a statistical model that predicts correctly the class of each sample. This training set is then tested with independent data (referred to as a test or validation set) to determine the robustness of the computer-based model. A machine learning model as described herein may comprise an ensemble of models whose predictions are combined. Alternatively, a machine learning model may comprise a single model. Supervised methods can use a data set with reduced dimensionality (for example, the first few principal components), but typically use unreduced data, with all dimensionality. The robustness of the predictive models can also be checked using cross-validation, by leaving out selected samples from the analysis. Any classification algorithm may be used in accordance with the present disclosure, including for example a regression model, k-nearest neighbour classifier, naïve Bayes classifier, etc. The machine learning model may be a regression model, i.e. a model that captures the relationship between a dependent variable (the variables that are being predicted) and a set of independent variables (also referred to as predictors). Any machine learning regression model may be used according to the present invention. For example, a machine learning model may be a random forest regressor (RF), a support vector machine (SVM), a logistic regression model (LR), a generalised linear model with or without regularisation (such as e.g. a binomial generalised linear model with elastic-net regularisation, GLMEN), a decision tree, or a k-nearest neighbour regressor. As detailed in the Examples herein, logistic regression (LR), support vector machine (SVM), generalised linear models with elastic-net regularisation (GLMEN) and Random Forests (RF) were used for variable selection and the classification of samples as “healthy” or “cancer”. A random forest regressor is a model that comprises an ensemble of decision trees and outputs a class that is the average prediction of the individual trees. Decision trees perform recursive partitioning of a feature space until each leaf (final partition sets) is associated with a single value of the target. Regression trees have leaves (predicted outcomes) that can be considered to form a set of continuous numbers. Random forest regressors are typically parameterized by finding an ensemble of shallow decision trees. A logistic regression model (also referred to as “logit model”) is a statistical model that uses a logistic function to model a binary dependent variable. A support vector machine is an algorithm that identifies a hyperplane or set of hyperplanes which can be used for classification or regression. A generalized linear model is a generalization of linear regression in which the response variable can have an error distribution that departs from a normal distribution. In particular each outcome of the dependent variables is assumed to be generated from a particular distribution in an exponential family (a class of distributions that includes the normal, Poisson and gamma distributions) whose mean depends on the independent variables. A regularized regression method is a process whereby additional constraints are provided to prevent overfitting, by introducing a regularization term or penalty that imposes a cost on the optimization function to make the optimal solution unique. The elastic net regularization method linearly combines penalties of the lasso (Tibshirani, Robert (1996). “Regression Shrinkage and Selection via the lasso”. Journal of the Royal Statistical Society. Series B (methodological). Wiley. 58 (1): 267-88) and ridge (see e.g. Gruber, Marvin (1998). Improving Efficiency by Shrinkage: The James-Stein and Ridge Regression Estimators. Boca Raton: CRC Press. pp. 7-15. ISBN 0-8247-0156-9.) methods.

“Computer-implemented method” where used herein is to be taken as meaning a method whose implementation involves the use of a computer, computer network or other programmable apparatus, wherein one or more features of the method are realised wholly or partly by means of a computer program. The systems and methods described herein may be implemented in a computer system, in addition to the structural components and user interactions described. As used herein, the term “computer system” includes the hardware, software and data storage devices for embodying a system or carrying out a method according to the above described embodiments. For example, a computer system may comprise a processing unit, such as a central processing unit (CPU) and/or a graphics processing unit (GPU), input means, output means and data storage, which may be embodied as one or more connected computing devices. Preferably the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer. The methods described herein may be provided as computer programs or as computer program products or computer readable media carrying a computer program which is arranged, when run on a computer, to perform the method (s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Analysis of a Urine Sample

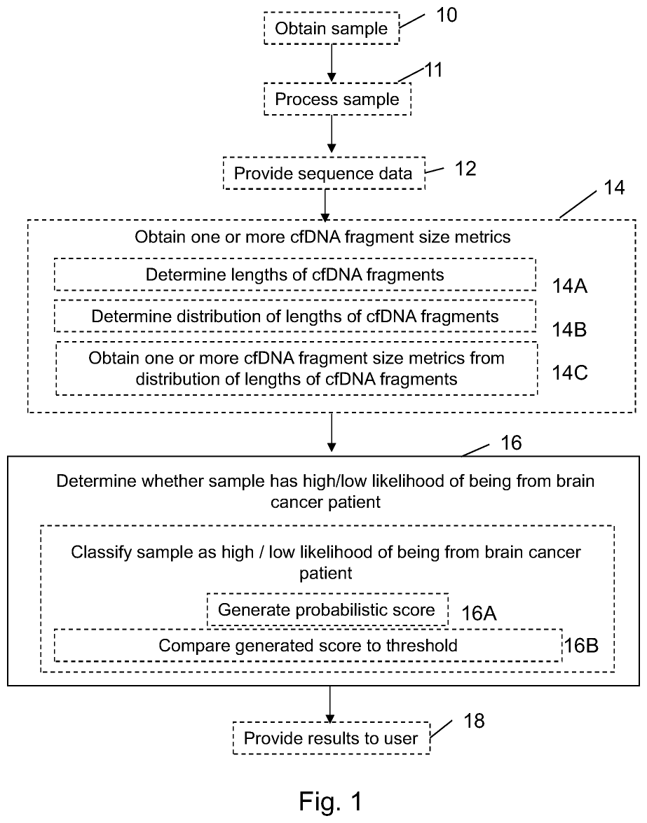

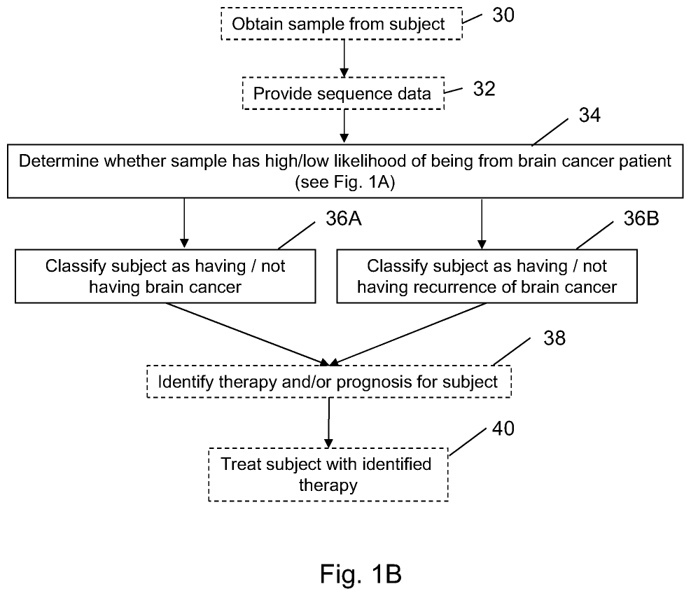

FIG. 1A illustrates a method for analysing a urine sample according to the disclosure. The method may comprise optional step 10 of obtaining a urine sample from a patient, optional step 11 of processing said sample, optional step 12 of providing sequence data from said sample, and optional step 14 of obtaining the value of one or more cfDNA fragment size metrics. Alternatively, the sample may have been previously obtained and/or processed to obtain sequence data, and the method may start using sequence data or values derived therefrom, such as the values of one or more cfDNA fragment size metrics as described herein. Processing the sample at step 11 may comprise steps of storing the sample, preserving the sample (e.g. refrigerating, freezing, otherwise processing to prevent damage such as e.g. by adding EDTA), purifying the sample (for example removing cells and debris e.g. by centrifugation), extracting DNA from the sample, extracting cfDNA or enriching the sample for cfDNA, for example by size selection to remove genomic DNA, etc. The step of providing sequence data may comprise sequencing DNA from said urine sample, or a library derived therefrom. The step of obtaining cfDNA fragment size metrics may comprise a step 14A of determining the lengths of cfDNA fragments from the sequence data, for example by aligning reads (e.g. paired end reads) in the sequence data to a suitable reference genome and determining the length of the sequence between the two ends of each fragment. At step 14B, a distribution of lengths of cfDNA fragments may be obtained based on the lengths determined at step 14B, for example in the form of a density function. At step 14C the value of one or more cfDNA fragment size metrics is/are obtained from the distribution of lengths of cfDNA fragments, for example by quantifying the proportion of fragments within one or more size ranges and/or by quantifying the amplitude of oscillations within a predetermined size range as described herein. At step 16, it is determined whether the sample has a high or low likelihood of being from a cancer patient, based on the values of the one or more metrics obtained at step 14. This can be performed by classifying the sample between at least two classes using a machine learning model, one class being associated with a high likelihood of being from a cancer patient and one class being associated with a low likelihood of being from a cancer patient. This can be performed for example by generating a probabilistic score at step 16A, and comparing this score to a threshold at step 16B. A probabilistic score may for example be indicative of a likelihood of being from a cancer patient (e.g. when the machine learning model used in step 16 is a regression model such as a logistic regression model), or may be indicative of the confidence of classification in a category associated with a high likelihood of being from a cancer patient (e.g. when the machine learning model used in step 16 is a support vector machine or random forest). The threshold used at step 16B may have been obtained as one of the parameters of the machine learning model during training of the model, as a threshold that results in the most accurate classification of training samples. At optional step 18, one or more results of any of the preceding steps may be provided to a user, for example via a user interface.

### Use of Analysis Outcome

The methods described herein find use in detecting the presence of, growth of, prognosis of, regression of, residual disease, treatment response of, or recurrence of a brain cancer in a subject, by analysing a urine sample from said subject. Each of these uses is based on the highly accurate detection of cancer-associated patterns in the pool of cfDNA molecules in urine samples using the methods described herein, which are in particular able to discriminate between samples from brain cancer patients and samples from patients without a brain cancer (including healthy patients and patients with other central nervous system diseases).

FIG. 1B illustrates a method for providing a diagnosis, prognosis or treatment recommendation according to the disclosure. The method may comprise obtaining a urine sample from a subject at step 30, and providing sequence data from said sample at step 32. Alternatively, the sample may have been previously obtained and/or processed to obtain sequence data, and the method may start using sequence data or values derived therefrom, such as the values of one or more cfDNA fragment size metrics as described herein. At step 34, it is determined whether the sample has a high or low likelihood of being from a brain cancer patient. At step 36A, a patient may be diagnosed as having brain cancer, for example if the patient has not been previously diagnosed as having brain cancer and/or if the subject is suspected of having brain cancer. At step 36B, a patient may be identified as having/not having a recurrence of a brain cancer, for example if the patient has been previously diagnosed as having brain cancer and the cancer has been treated. Steps 30-36 may be repeated a number of times, for example for longitudinal monitoring of a subject who is identified as likely to develop a brain cancer or a subject who has been treated for brain cancer (e.g. to monitor regression, residual disease and/or recurrence). At optional step 38, a therapy and/or prognosis may be identified for the subject depending on the outcome of step 36A/36B. For example, a subject classified as having brain cancer at step 36A or likely to have recurrence at step 36B may be selected for (further) cancer therapy or identified as likely to have poor prognosis. Further, the confidence of the classification at step 36A/36B may be indicative of prognosis and/or may guide the therapeutic strategy. For example, a classification with low confidence may prompt further diagnosis (e.g. invasive diagnostic tests or imaging). As another example, a classification with very high confidence (e.g. high likelihood, compared to medium or low likelihood) may be indicative of a strong ctDNA signal, possibly correlating with larger amounts of ctDNA in the sample and hence poor prognosis/stronger cause for therapeutic intervention. At optional step 40, the subject may be treated with a cancer therapy for which the subject has been selected at step 38.

Whether a prognosis is considered good or poor may vary between cancers and stage of disease. In general terms a good prognosis is one where the overall survival (OS), disease free survival (DES) and/or progression-free survival (PFS) is longer than that of a comparative group or value, such as e.g. the average for that stage and cancer type. A prognosis may be considered poor if OS, DES and/or PFS is lower than that of a comparative group or value, such as e.g. the average for that stage and type of cancer. Thus, in general terms, a “good prognosis” is one where survival (OS, DES and/or PFS) and/or disease stage of an individual patient can be favourably compared to what is expected in a population of patients within a comparable disease setting. Similarly, a “poor prognosis” is one where survival (OS, DFS and/or PFS) of an individual patient is lower (or disease stage worse) than what is expected in a population of patients within a comparable disease setting.

### Systems

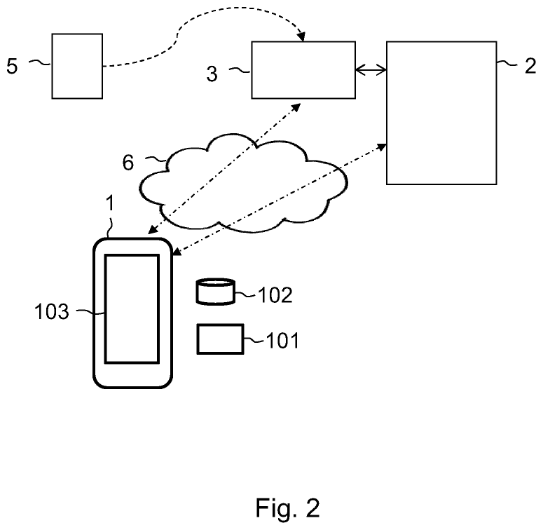

FIG. 2 shows an embodiment of a system for analysing a urine sample and/or for providing a diagnosis, prognosis, treatment recommendation or monitoring according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network, to sequence data acquisition means 3, such as a sequencing machine, and/or to one or more databases 2 storing sequence data. The one or more databases 2 may further store one or more of: training data, parameters (such as e.g. parameters of a machine learning model used to predict whether sample is from a brain cancer patient, e.g. weights of a logistic regression model, architecture and parameters of a decision tree model, etc.), clinical and/or sample related information, reference genome information, etc. The computing device may be a smartphone, tablet, personal computer or other computing device. The computing device is configured to implement a method for analysing a urine sample, as described herein. In alternative embodiments, the computing device 1 is configured to communicate with a remote computing device (not shown), which is itself configured to implement a method of analysing a urine sample, as described herein. In such cases, the remote computing device may also be configured to send the result of the method of analysing a urine sample to the computing device. Communication between the computing device 1 and the remote computing device may be through a wired or wireless connection, and may occur over a local or public network 6 such as e.g. over the public internet. The sequence data acquisition means may be in wired connection with the computing device 1, or may be able to communicate through a wireless connection, such as e.g. through WiFi and/or over the public internet, as illustrated. The connection between the computing device 1 and the sequence data acquisition means 3 may be direct or indirect (such as e.g. through a remote computer). The sequence data acquisition means 3 are configured to acquire sequence data from nucleic acid samples, for example genomic DNA samples extracted from cells and/or tissue samples. The system may further comprise a device 5 for collection and/or processing of a urine sample. In some embodiments, the sample may have been subject to one or more preprocessing steps such as DNA purification, fragmentation, library preparation, size selection, etc. Any of these steps may be performed by the device 5. Once a sample of cfDNA has been obtained, for example through use of the device 5, the sample may be provided as input to the sequence data acquisition means 3. Preferably, the sample has not been subject to amplification, or when it has been subject to amplification this was done in the presence of amplification bias controlling means such as e.g. using unique molecular identifiers. Any sample preparation process that is suitable for use in the determination of the size distribution of cfDNA fragments (whether whole genome or sequence specific) may be used within the context of the present invention. The sequence data acquisition means is preferably a next generation sequencer.

The following is presented by way of example and is not to be construed as a limitation to the scope of the claims.

### EXAMPLES

### Materials and Methods

### Study Design

A total of 35 glioma patients (30 high grade glioma HGG, 5 low grade glioma LGG) were recruited. Among the 5 LGG, 3 were diffuse astrocytoma, 1 was an oligodendroglioma and 1 a pilocytic astrocytoma. Among the 30 HGG, 29 were glioblastomas (GBM) and 1 was an anaplastic oligodendroglioma (AO). Matched tumour tissue, CSF, plasma, urine and buffy coat samples were collected for 8 patients. In addition, urine samples were collected from 26 healthy volunteers and 27 patients with other pathologies of the brain or central nervous system (CNS). Body fluid samples were analysed using two sequencing based approaches: patient-specific hybrid capture panels, and sWGS (shallow whole genome sequencing).

### Sample Collection and Preparation

Lumbar puncture was performed immediately prior to craniotomy for tumour debulking. After sterile field preparation, the thecal sac was cannulated between the L3 and L5 intervertebral spaces using a 0.61 mm gauge lumbar puncture needle, and 10 ml of CSF was removed. After collection, CSF, whole blood and urine samples were immediately placed on ice and then rapidly transferred to a pre-chilled centrifuge for processing. For urine samples, 0.5M EDTA was added within an hour of collection. Samples were centrifuged at 1500 g at 4° C. for 10 minutes. Supernatant was removed and further centrifuged at 20,000 g for 10 minutes, and aliquoted into 2 mL microtubes for storage at −80° C. (Sarstedt, Germany). Tumour tissue DNA were extracted and isolated as described previously (Mouliere et al, 2018b). Fluids were extracted using the QIAsymphony platform (Qiagen, Germany). Up to 10 mL of plasma, 10 mL of urine and 8 mL of CSF was used per sample. DNA from cancer plasma, urine and CSF samples was eluted in 90 μL, and further concentrated down to 30 μL using a Speed-Vac concentrator (Eppendorf, Germany).

### Sequencing Library Preparation and WES for Tissue DNA

In order to identify patient specific somatic mutations, the inventors first performed whole exome sequencing (WES) of all tumour tissue and germline buffy coat DNA samples. Fifty nanograms of DNA were fragmented to ˜120 bp by acoustic shearing (Covaris) according to the manufacturer's instructions. Libraries were prepared using the Thruplex DNA-Seq protocol (Rubicon Genomics) with 5× cycles of PCR. Libraries were quantified using quantitative PCR (KAPA library quantification, KAPA biosystems) and pooled for exome capture (TruSeq Exome Enrichment Kit, Illumina). Exome capture was performed with the addition of i5 and i7 specific blockers (IDT) during the hybridization steps to prevent adaptor ‘daisy chaining’. Pools were concentrated using a SpeedVac vacuum concentrator (Eppendorf, Germany). After capture, 8× cycles of PCR were performed. Enriched libraries were quantified using quantitative PCR (KAPA library quantification, KAPA Biosystems), DNA fragment sizes were assessed by Bioanalyzer (2100 Bioanalyzer, Agilent Genomics) and captures were pooled in equimolar ratio for paired-end next generation sequencing on a HiSeq4000 (Illumina). Sequencing reads were de-multiplexed, allowing zero mismatches in barcodes. The reference genome was the GRCh37/b37/hg19 human reference genome—Genomes GRCh37-derived reference genome, which includes chromosomal plus unlocalized and unplaced contigs, the rCRS mitochondrial sequence (AC: NC_012920), Human herpesvirus 4 type 1 (AC:NC 007605) and decoysequence derived from HuRef, Human Bac and Fosmid clones and NA12878. The sequence data of the patient samples were aligned to the reference genome using BWA-MEM v0.7.15. The duplicate reads were marked using Picard v1.122 (http://broadinstitute.github.io/picard). Somatic SNV and indel mutations were called using GATK Mutect2 (Genome Analysis Toolkit), (https://www.broadinstitute.org/gatk) in tumour-normal pair mode using buffy coat as the normal. MAFs for each single-base locus were calculated with MuTect2 for all bases with PHRED quality 230. After MuTect2, we applied filtering parameters so that a mutation was called if no mutant reads for an allele were observed in germline DNA at a locus that was covered at least 10×, and if at least 4 reads supporting the mutant were found in the tumour data with at least 1 read on each strand (forward and reverse). Variants were annotated using Ensembl Variant Effect Predictor with details about consequence on protein coding, accession numbers for known variants and associated allele frequencies from the 1000 Genomes project.

### Tumour-Guided Capture Sequencing

Hybrid-based capture for the different body fluids (CSF, plasma, urine) analysis was designed to cover the variants identified above for each patient using the SureDesign software (Agilent). In addition, 52 genes of interest for glioma were included in the tumor-guided sequencing panel based on the TCGA databases. Patients were separated into 2 panels covering all the mutations included for those patients (4 patients per panel). Panel 1 covered in total 526 kbp (5841 regions) and panel 2 covered 526 kbp (5701 regions). Panels ranged in size between 1.430 Mb (panel 1) and 1.404 Mb (panel 2) with 120 bp RNA baits. Baits were designed with 5× tiling density, moderately stringent masking and balanced boosting. 99.7% of the targets had baits designed successfully. Indexed sequencing libraries were prepared using the Thruplex tag-seq kits (Takara). Libraries were captured either in 1-plex for plasma and urine samples or 3-plex for CSF samples (to a total of 1000 ng capture input) using the Agilent SureSelectXTHS protocol, with the addition of i5 and i7 blocking oligos (IDT), as recommended by the manufacturer for compatibility with ThruPLEX libraries. Custom Agilent SureSelectXTHS baits were used. 13 cycles were used for amplification of the captured libraries. Post-capture libraries were purified with AMPure XT beads, then quantified using quantitative PCR (KAPA library quantification, KAPA Biosystems), and DNA fragment sizes controlled by Bioanalyzer (2100 Bioanalyzer, Agilent Genomics). Capture libraries were then pooled in equimolar ratios for paired end next generation sequencing on a HiSeq4000 (Illumina).

### Capture Sequencing Analysis

Sequencing reads were de-multiplexed, allowing zero mismatches in barcodes. Cutadapt v1.9.1 was used to remove known 5′ and 3′ adaptor sequences specified in a separate FASTA 640 of adaptor sequences. Trimmed FASTQ files were aligned to the UCSC hg19 genome using BWA-mem v0.7.13 with a seed length of 19. Error suppression was carried out on ThruPLEX Tag-seq library BAM files using CONNOR. The consensus frequency threshold (−f) was set as 0.9 (90%), and the minimum family size threshold (−s) was varied between 2 and 5 for characterization of error rates (Wan et al, 2020). Patient-specific sequencing data consists of informative reads at multiple known patient-specific loci that were identified from tumour sequencing (see above).

sWGS

Indexed sequencing libraries were prepared using the ThruPLEX-Plasma Seq kit (Rubicon Genomics). Libraries were pooled in equimolar amounts and sequenced to <0.4× depth of coverage on a HiSeq 4000 (Illumina) generating 150-bp paired-end reads. Sequence data was analysed using an in-house pipeline that consists of the following steps. Paired end sequence reads were aligned to the human reference genome (GRCh37) using BWA-mem following the removal of contaminating adapter sequences. PCR and optical duplicates were marked using MarkDuplicates (Picard Tools) feature and these were excluded from downstream analysis along with reads of low mapping quality and supplementary alignments. When necessary, reads were down-sampled to 10 million in all samples for comparison purposes.

### Fragmentation Feature Analysis

The preliminary analysis was carried out on 93 samples (40 cancers and 53 noncancer controls). For each sample the following features were calculated from sWGS data: P(30-60), P(61-90), P(91-120), P (121-150), 690 P(151-180), P(181-210), P(211-240), P(241-270), P (271-300). The data was arranged in a matrix where the rows represent each sample and the columns held the aforementioned features with an extra “class” column with the binary labels of “cancer” or “controls”. The amplitude of the 10 bp periodic peaks (OSC_10 bp) was calculated from the sWGS data as follows: from the samples with clear peaks, the local maxima (“peak”) and minima (“valley”) in the range 50-140 bp were calculated. The average of their positions across the samples was calculated: (minima: 62, 73, 84, 96, 106, 116, 126, 137; and maxima: 58, 69, 80, 92, 102, 112, 122, 134). To compute the “amplitude statistic”, the inventors calculated the sum of the height of the maxima and subtracted the sum of the height of the minima. The larger this difference, the more distinct are the peaks. The height of the x bp peak is defined as the number of fragments with length x divided by the total number of fragments. To define local maxima, the inventors selected the positions y such that y was the largest value in the interval [y−2, y+2]. The same rationale was used to pick minima. PCA were calculated and visualized in R using the package ggbiplot. The tSNE analysis was performed in R with the Rtsne package using 1000 iterations, Spearman correlations and a perplexity score of 8. Plots were generated in R using ggplot2. ROC curves were plotted in R with the plotROC package.

### Predictive Analysis

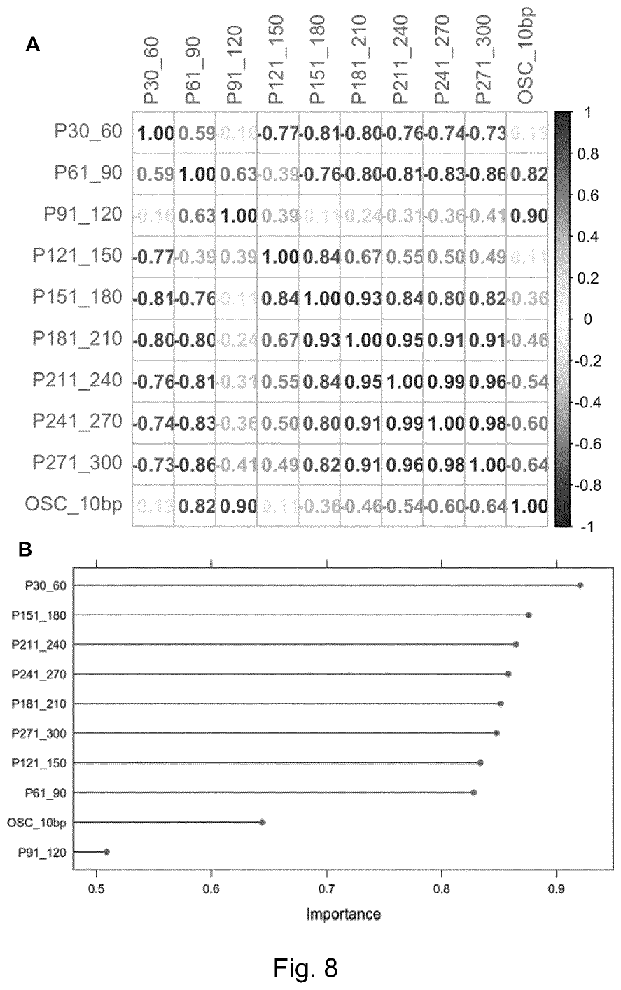

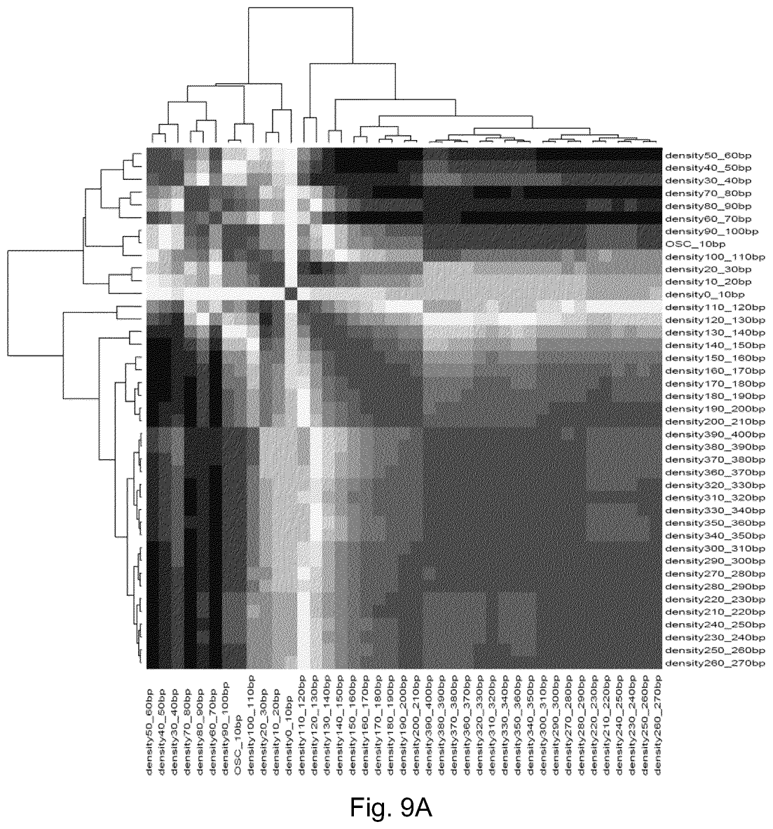

The following analysis was carried out in R utilising RandomForest, and pROC packages and in Python using scikit-learn and H2O Python API modules. The pairwise correlations between the features were calculated to assess multi-collinearity in the dataset (FIG. 8A). Feature importance was analysed and quantified using a LVQ model. The algorithm was configured to explore all possible subsets of the features. After this pre-processing all the 10 features were retained for further analysis. The data matrix for the 93 samples (40 cancer samples and 53 controls) were randomly partitioned into five batches of comparable size, four of which were used for training and one was used for testing (80:20 split). For every cross validation, baseline and follow-up samples of the same patient were randomly distributed in the training set or in the test set. In each of the resulting 5 folds, the training set was split once more using stratified 5-fold cross-validation. This cross validation scheme was repeated for 10 iterations, yielding 50 iterations in total. Classification of samples as healthy or cancer was performed using logistic regression (LR), random forest (RF), support vector machine (SVM) and binomial generalized linear models with elastic-net regularization (GLMEN). Predictions on the test set were stored for each of the models 50 folds. To evaluate the performance metric of the models, a ROC curve was calculated for each fold validation and a mean ROC curve were then calculated based on these 50 curves. Mean performance over 50 iterations for precision, recall, accuracy, sensitivity, specificity were also calculated for each model, and in various scenarios (by selecting all samples, only baseline samples, all features, only 4 features).

### Statistical Analysis

All statistics were performed using R (v3.4.3) programming language (www.rproject.org). We also used the ggplot2 (v3.2.0) and ggpubr (v0.2) packages.

### Data Availability

Raw sequencing data is deposited at the European Genome-phenome archive, (https://ega-740 archive.org/studies/EGAS00001004355).

### Example 1: Tumour-Derived cfDNA Fragments are Shorter than Non-Mutant cfDNA in the CSF, Plasma and Urine Samples of Glioma Patients

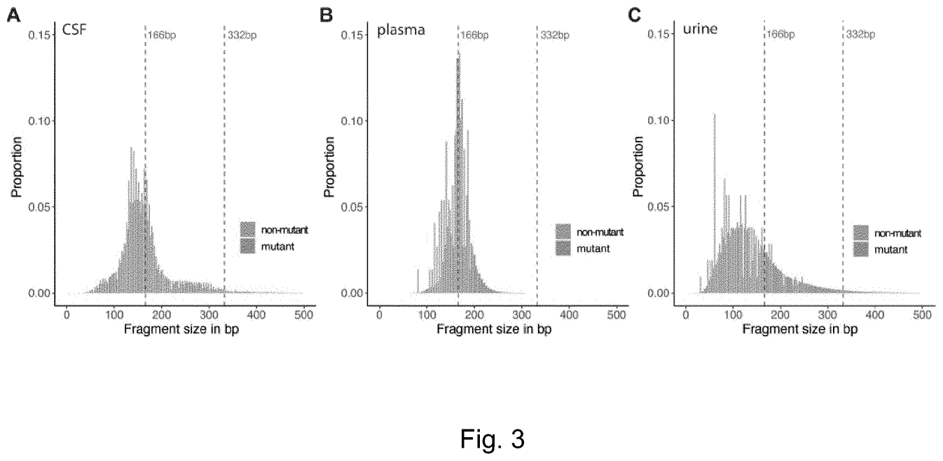

Using paired-end sequencing reads from hybrid capture panels (targeting the 52 most frequently mutated genes in Glioma (Brennan et al., 2013) and single nucleotide variants identified by comparing tumour and non-tumour sequences in 8 glioma patients), the inventors determined the distribution of read lengths (fragmentation patterns) of mutant and non-mutant cfDNA, i.e. reads carrying mutations previously identified in matched tissue and those not carrying mutations, in the CSF (FIG. 3A), plasma (FIG. 3B) and urine (FIG. 3C) of the 8 glioma patients pre-surgery. Reads carrying tumour-identified mutations represent cfDNA fragments that are highly likely to be derived from the tumour DNA, whereas those without a tumour-identified mutation likely represent a mixture of non-tumour DNA, and non-mutated DNA copies from tumour cells. The use of error suppression in the sequencing data analysis results in minimal levels of noise (Wan et al, 2020). In the 3 bio-fluids, the inventors observed a consistent and significant shift towards shorter fragment sizes for mutant cfDNA in comparison to non-mutant cfDNA: in CSF samples, median size of 148 bp for mutant cfDNA vs 169 bp for non-mutant cfDNA; in plasma samples, 160 bp vs 169 bp; and in urine samples, 101 bp vs 133 bp (two-sided Wilcoxon, p<0.0001 for all three body fluids). fluids). Such a shift was described previously for plasma samples of other cancer types (Mouliere et al, 2011; Underhill et al, 2016; Mouliere et al, 2018a; van der Pol & Mouliere, 2019), but has not previously been observed directly in the urine and CSF of patients with gliomas, or other malignancies, by analysis of specifically mutant-derived fragments. The inventors hypothesized that, in a similar way to their previous observations in plasma (Mouliere et al, 2018a), the size difference observed in urine could be identified using more scalable methods, to improve ctDNA detection in this non-invasive liquid biopsy without requiring tumour tissue DNA analysis.

### Example 2: Analysis of cfDNA Fragmentation Patterns in Urine by Shallow Whole Genome Sequencing

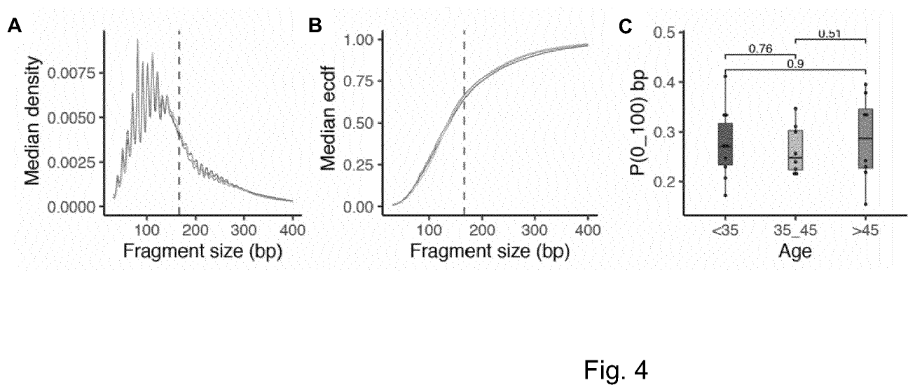

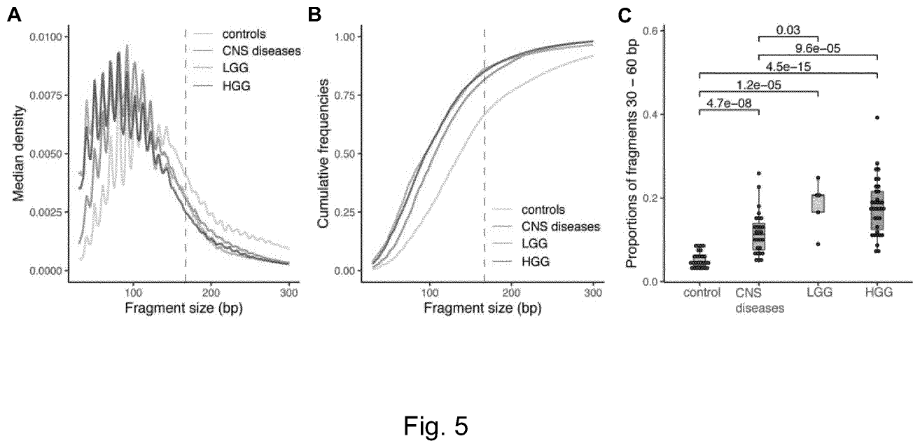

The inventors analysed the cfDNA fragmentation patterns in 40 urine samples from 35 patients with gliomas (30 HGG and 5 LGG) collected pre-treatment with paired-end sWGS. They also sequenced urine cfDNA from 53 controls: 26 healthy individuals and 27 patients with other pathologies affecting the central nervous system (cervical myelopathy, cerebral artery aneurysm-both ruptured and unruptured, hydrocephalus and Parkinson's disease). Baseline urine samples from patients with cancer and other CNS pathologies were collected prior to surgery, and follow-up samples were collected for a subset of the cases. Age and other physiological properties of the cases and controls were collected. All urine samples were collected and processed according to the same protocol and time-frame for processing to reduce potential biases due to differences in pre-analytical processing (see Materials and Methods). The mean age of the healthy individuals was lower than for the cancer cases (41 years old and 61 years old, respectively). The inventors therefore evaluated the influence of donor age on the cfDNA fragment size distribution of the cohort of healthy individuals, and observed no significant difference (FIG. 4). Of note, the concentration of cfDNA extracted from urines increased from a mean of 4.25 ng/ml in controls to 10.1 ng/ml in glioma patients. The cfDNA median size distribution in the urine of healthy individuals was 137 bp, 108 bp in the urine of patients with other brain or CNS pathologies, and 101 bp in the urine of glioma patients (FIG. 5A). cfDNA in urine of glioma patients was significantly shorter and more fragmented than in urine of healthy individuals (FIG. 5B) (Wilcoxon, p=5.2×10−9), and in urine of patients with other brain pathologies (Wilcoxon, p=1.7×10−2). The inventors calculated the median empirical cumulative distribution function for each type of sample included in the study (FIG. 5B). The cumulative distribution indicated that the median fragment size distribution of HGG was significantly different to that of healthy controls (Kolmogornov-Smirnov, distance=0.476, p<0.001), and of other CNS pathologies (Kolmogornov-Smirnov, distance=0.287, p<0.001). The inventors analysed the proportion of fragments in different size ranges, and observed that the proportion of fragments between 30-60 bp was significantly increased in HGG and LGG cases as compared to healthy controls (Wilcoxon, p<0.001 for HGG and p<0.001 for LGG) and was also increased when compared to patients with other brain or CNS pathologies (Wilcoxon, p<0.001 for HGG and p=0.03 for LGG), (FIG. 5C).

### Example 3: Leveraging Fragmentation Patterns of Urine cfDNA for Classification of Glioma Patients from Controls

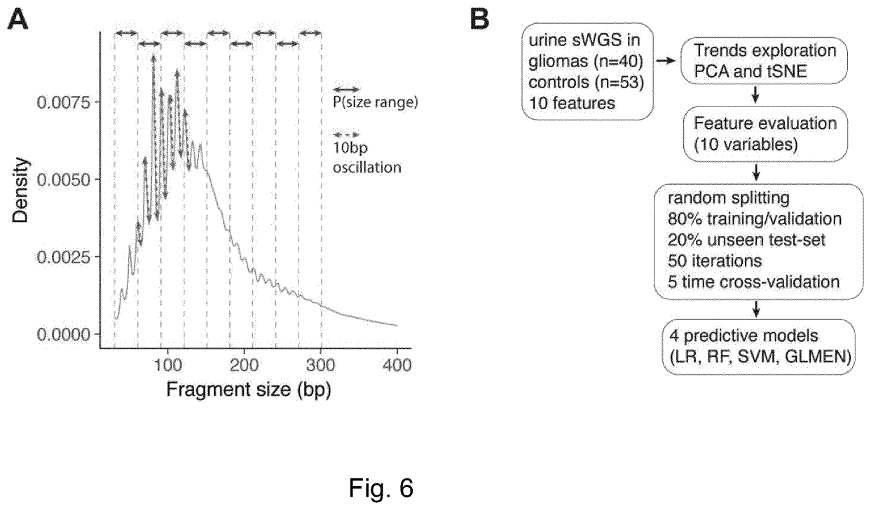

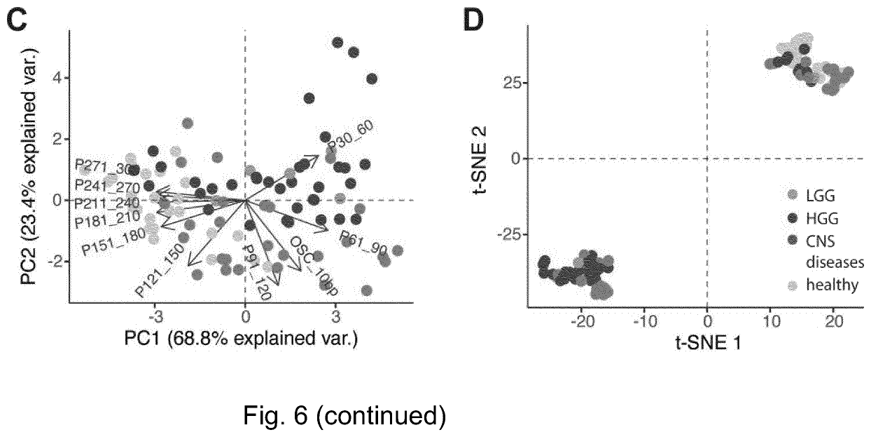

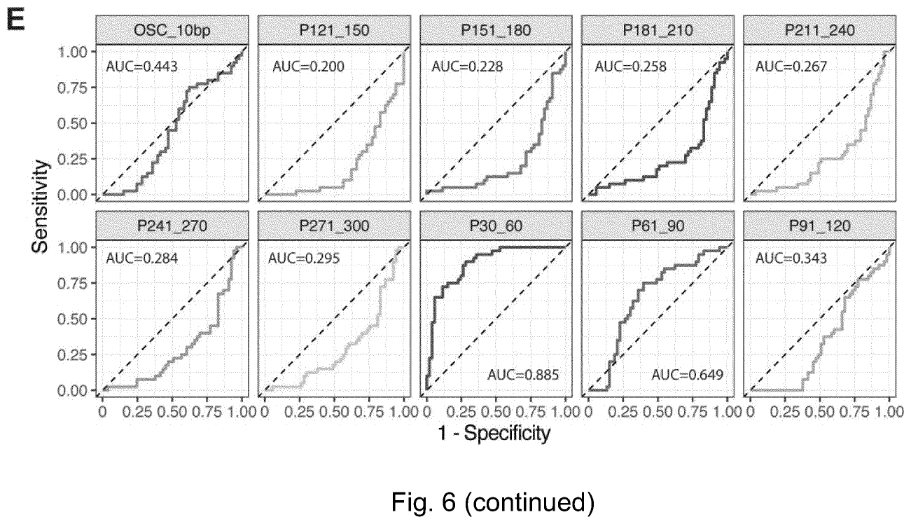

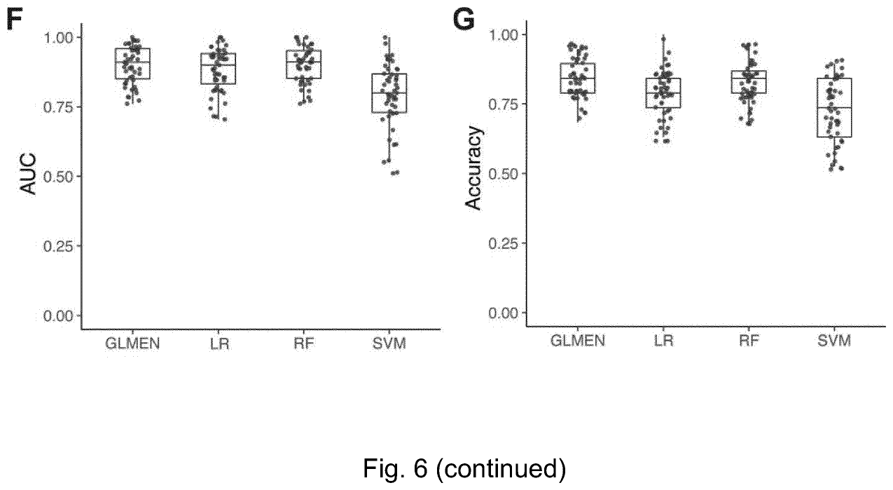

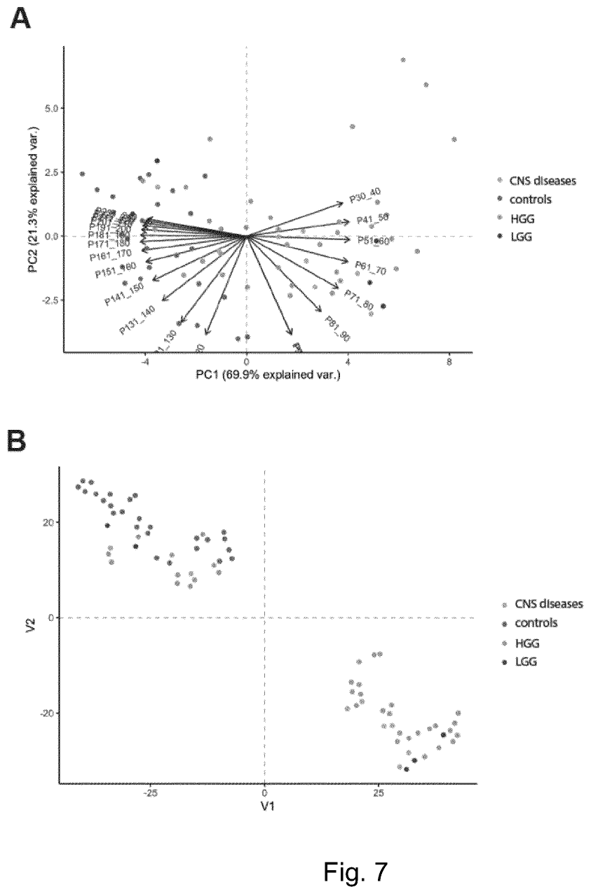

The inventors demonstrated previously that cfDNA fragmentation features could be used to improve the detection of glioma in plasma samples (Mouliere et al, 2018a). In plasma samples, a random forest model comprising a copy number-based feature (t-MAD), and 4 fragment size features (OSC10, p(160-180), p(180-220), p(250-320), respectively the amplitude of 10 bp peaks (oscillations) in the distribution of fragment lengths in the 75-150 bp range, the proportion of fragments in the 160-180 bp, 180-220 bp and 250-320 bp range) was found to perform best at distinguishing cancer vs healthy samples. Here they explored whether these features in urine could be used to enhance detection of tumour DNA in glioma patients, and further to enable this detection in the presence of confounding factors such as the influence of the possible presence of other CNS disease on the cfDNA fragmentation profile. A predictive analysis was performed using 10 fragmentation features across 93 urine samples (40 samples from 35 cancer cases and 53 samples from 53 non-cancer controls). These ten fragmentation features were based on the proportion (P) of fragments in the following size ranges in sWGS data from each sample, using 30 bp bins: P(30 to 60), P (61 to 90), P (91 to 120), P(121 to 150), P (151 to 180), P(181 to 210), P (210 to 240), P (241 to 270) and P (271 to 300) (FIG. 6A and FIG. 6B). The last feature corresponds to the 10 bp peaks (oscillations) in the distribution of fragment lengths, which have been reported previously (Mouliere et al, 2018a, 2018b) and are particularly pronounced in urine samples (note that in this case this metric was calculated in the 50-140 range rather than the 75-150 range used in plasma, reflecting the different fragmentation profile observed in urine compared to plasma). The inventors demonstrated clustering of the data using principal component analysis (PCA) (FIG. 6C) and t-distributed stochastic neighbour embedding (tSNE) (FIG. 6D). These indicated that a higher proportion of shorter fragments (<91 bp) could be indicative of cancer samples (FIG. 6C and FIG. 6D). The inventors performed k-means clustering, assuming k=2, and identified a cluster with 29 data-points consisting of a high proportion of cancer samples (n=27/29, 94% cancer samples), and a second cluster with 45 data points and a mixture of non-cancer and cancer samples (n=13/45, 28% cancer samples). Analysis of cfDNA fragments using 10 bp bin sizes showed less pronounced clustering (FIG. 7A and FIG. 7B). The inventors tested the individual features and calculated a binary classification to separate “cancer” (HGG and LGG) from “control” samples (healthy and other CNS disease controls) (FIG. 6E). The feature P30_60 (the proportion of fragments between 30 and 60 bp in length) exhibited the highest classification performance (AUC=0.885).

Variable selection and the classification of samples as “non-cancer” or “cancer” were performed using logistic regression (LR) and other machine learning models trained and validated on 40 cancer samples and 53 controls (FIG. 8 and FIG. 6B). The performance of the models was evaluated for using the 10 feature sets, using a double cross-validation scheme and 50 random bootstrap iterations (see Materials and Methods) (FIG. 6B). Using the SVM model the inventors could distinguish non-cancer from cancer samples with a median AUC=0.80 (range 0.51-1) (FIG. 6F and FIG. 6G). Sensitivity analyses considering other machine learning methods as classifiers led to similar results in terms of AUC. The inventors compared random forest (RF), support vector machine (SVM) and a binomial generalized linear model with elastic-net regularization (GLMEN) to the LR model. Using the GLMEN model they could distinguish non-cancer from cancer samples with a median AUC=0.91 (range 0.76-1) (FIG. 6F) and a median accuracy=0.84 (range 0.68-0.95) (FIG. 6G). The RF model exhibited a median AUC=0.91 (range 0.76-1) and median accuracy=0.84 (range 0.68-0.94) (FIG. 6F and FIG. 6G). The LR model exhibited a median AUC of 0.9 (range 0.70-1) and accuracy=0.78 (range 0.63-1). Despite the small cohort size (n=93), which might affect the reproducibility of the models with an independent dataset, these results suggest that the cfDNA fragmentation patterns in urine samples may be a useful tool to provide information that can aid in the diagnosis of gliomas.

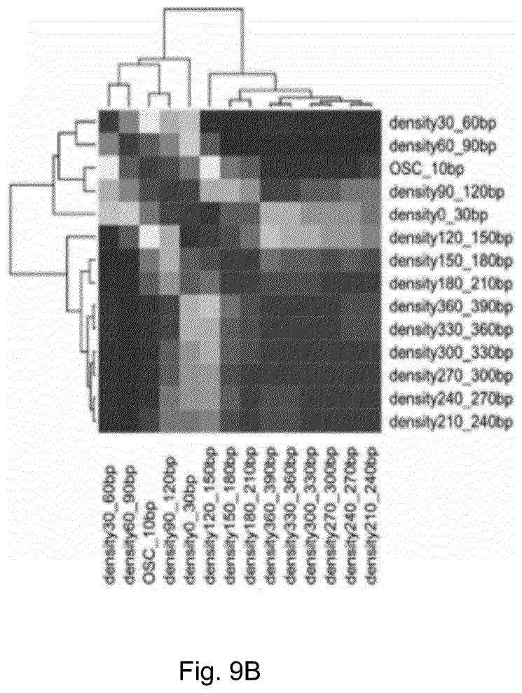

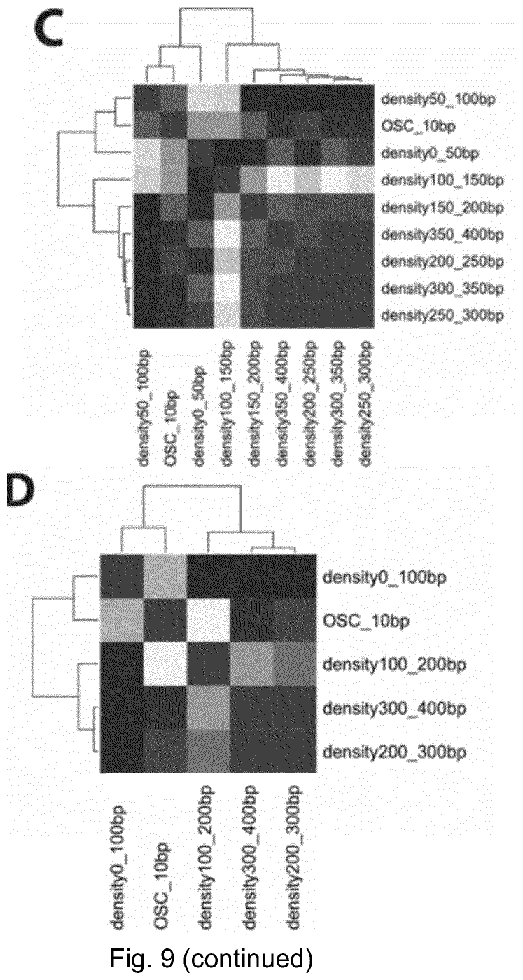

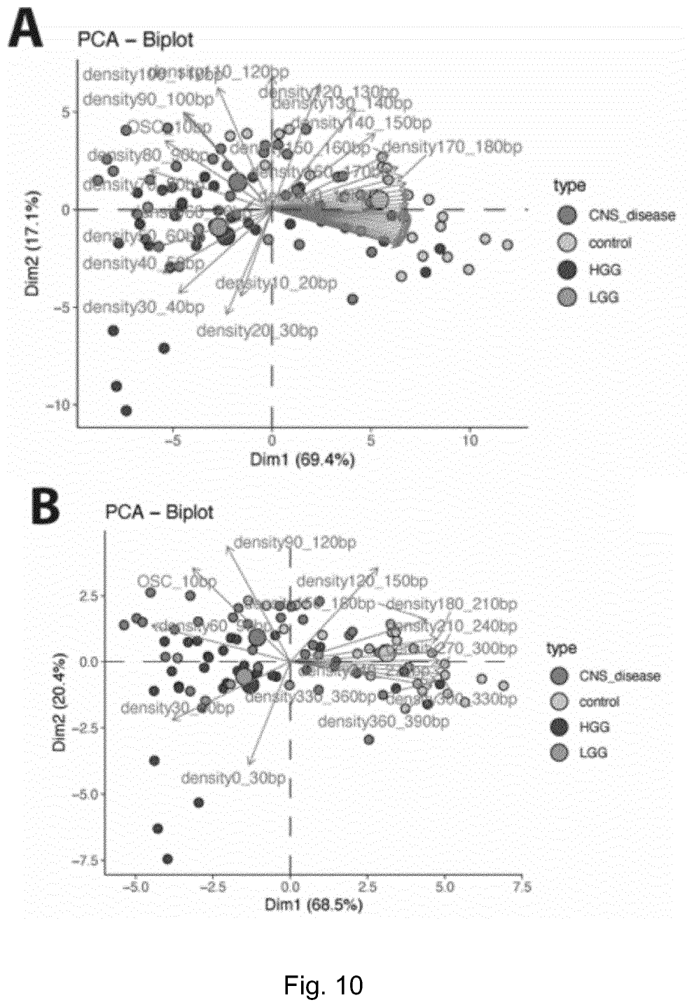

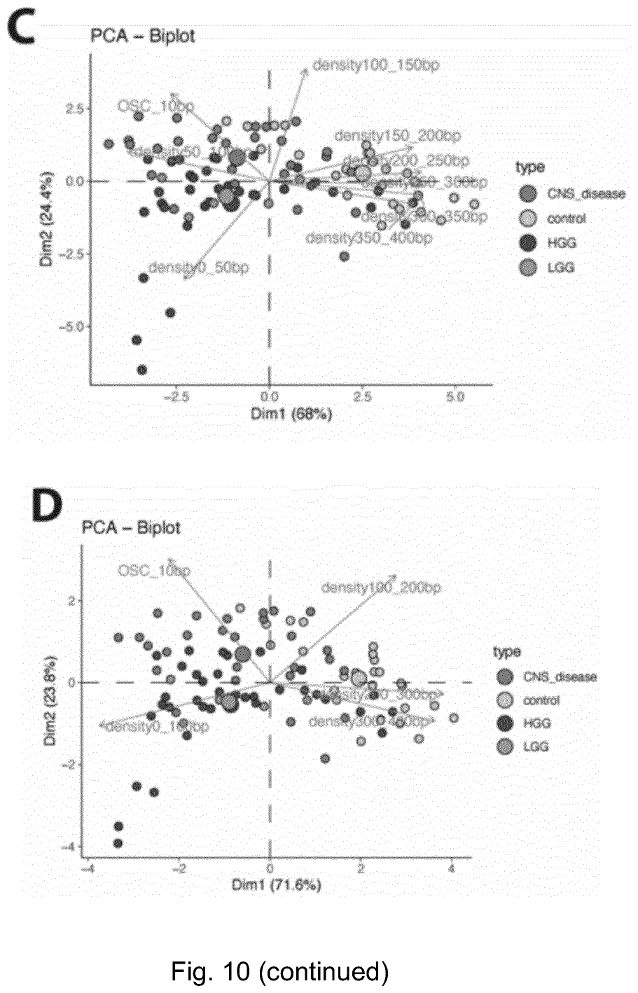

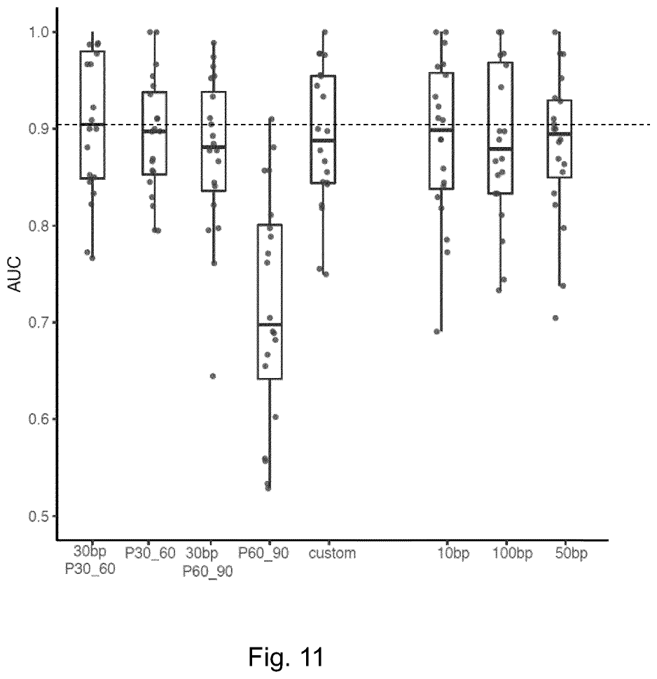

In order to better understand the information that can be obtained from fragment size features, the inventors evaluated the cross-correlations of features in the set of samples (40 cancers-HGG and LGG, 55 controls-healthy and non-cancer) (FIG. 9). The inventors used four different size feature binning strategies for this: (1) 10 bp bins across the range from 10 to 350 bp (FIG. 9A), (2) 30 bp bins across the range from 0 to 390 bp (FIG. 9B), (3) 50 bp bins across the range from 0 to 400 bp (FIG. 9C), and (4) 100 bp bins across the range from 0 to 400 bp (FIG. 9D). The data on FIG. 9D indicates that the 0-100 bp range provides different information from the 100-400 bp range, and that the information in the 300-400 bp range is largely redundant with the information in the 200-300 bp range. Thus, this indicates that an informative binning strategy could stop at 300 bp, and would likely capture information from the 0-100 bp range separately from information from 100 bp and above. The data on FIG. 9C further confirms this picture, with the 50 bp bins between 200 and 400 bp all providing very similar information, and the 0-50 bp and 50-100 bp providing information that is not highly similar to the information provided by any other bin. This data further indicates that the 100-150 bp bin also provides information that is complementary to that provided by both the 0-100 bp range and the 150-200 bp range. Thus, this data indicates that a relatively granular capture of the 0-150 bp range is likely to be informative (e.g. more informative than an approach that captures substantially this entire range in one bin). This is confirmed by the data on FIG. 9B, which shows that all of the bins within this range (i.e. 0-30, 30-60, 60-90, 90-120 and 120-150) capture interesting variation, whereas the bins above 150 bp each capture information that is more similar to each other. In particular, this data indicates that the 30-60 and 60-90 capture similar but not identical information, which is different from that captured by the 90-120 bin. Of note, the 0-30 bp bin appears to correlate poorly with all other bins, potentially indicating that this range is relatively noisy. This may be at least partially because the mapping of sequencing data relating to very short fragments is typically of lower quality. Thus, this range may negatively impact classification by introducing noise (at least when using sequencing data as input). A similar picture appears when looking at 10 bp bins (FIG. 9A). This data further indicates that there may be diminishing returns (or even a risk of introducing noise/overfitting in the classification) by further increasing the granularity of the bins. For example, the 30-40, 40-50 and 50-60 bins appear to provide similar information. The 0-10, 10−20 and 20-30 bp bin appear to contribute some noise and the bins in the 90-110 interval provide similar information although this is noisier than when looking at the entire range (0-20 bp bins and 100-120 bins have low correlation with all other bins, and the 0-20 bp range is more similar to the 110−120 bp range than it is to closer ranges such as the 40-50 range). The inventors then evaluated the unsupervised clustering of the features using the same four different size feature binning strategies (FIG. 10). This data confirms that the separation of the 0-100 bp range into more granular ranges improves the separation of the samples (compare FIG. 10D (100 bp) bins with FIG. 10C (50 bp bins), where FIG. 10C also shows that the 0-50 and 50-100 bp vectors contribute differently to the first two principal components), whereas the same is not observed to the same extent for the 200-400 bp range and especially for the 300-400 bp range (compare FIG. 10D with FIG. 10C). Note in particular that the 0-50, 50-100 and 100-150 appear to contribute quite differently to the first two principal components, and seem to provide complementary information to separate the samples. Looking at the data on FIG. 10B (30 bp bins), it seems that all 30 bp ranges until 150 bp contribute differently to the first two principal components and help to separate the samples, with the bins from 150 to 390 bp contributing similarly to the first two principal components. FIG. 10A (10 bp bins) confirms this picture and further seems to indicate that the additional granularity does not seem to improve the separation of groups of samples compared to FIG. 10B. Finally, the inventors ran a LR model as described above (except that only 20 iterations of sample bootstrapping were performed for every model) with these sets of features, as well as modified versions thereof that aim to investigate the importance of the 30-60, 60-90, 60-90 and 90-150 bins in the 30 bp bin feature set. The AUC was calculated for each of these models and the results are shown on FIG. 11 (where “30 bp P30-60” refers to a model using all features of the 30 bp feature set apart from the 30-60 bin, “P30_60” only uses the 30-60 bin, “30 bp P60_90” uses all features of the 30 bp feature set apart from the 60-90 bin, “P60_90” only uses the 60-90 bin, “custom” uses all features of the 30 bp feature set except that it combines the data in the 20-150 bp range). Note that these numbers are not directly comparable to those reported above and on FIG. 6 because a different number of iterations was used, and no feature selection was applied, i.e. the models use all of the bins in their respective binning schemes (e.g. the 30 bp model uses all bins between 0 and 390 bp whereas the models for which performance is reported on FIG. 6 only use 30 bp bins between 0 and 300 bp). Thus, the data for the different models on FIG. 11 is only comparable to each other. Further, the results on this figure refer to small amounts of iterations and a relatively small amount of data, such that comparing models should be performed on the basis of all of the information available as discussed above and not strictly based on the indicative numbers provided here. Further, additional data on which the models could be trained and tested would likely further sharpen the picture seen in these dat. Nevertheless, the data indicates that a good performance (median AUC above 0.9) can still be obtained with a 30 bp model that does not include the 30-60 bin, possibly because information in other bins such as the 60-90 bin or bins at the other end of the scale (which are inversely correlated with the 30-60 bin) are able to compensate for the lack of the 30-60 bin. A good performance can also be obtained using the 30-60 bin alone, indicating that the bin contains a lot of information that is very useful to the classification observed, although the loss of this information can potentially be compensated by granular information from other bins. The performance of a 30 bp model that does not include the 60-90 bin is slightly lower, although the performance of the 60-90 bin alone is not as good as that of the 30-60 bin alone. This indicates that the 60-90 bin also provides information that is useful to the classification, and that although on its own it may not have quite the same discrimination power as the 30-60 bin, it may provide information the loss of which is less easily compensated by other bins (i.e. the information in this bin may contribute slightly less to the discrimination but this contribution may be less redundant). The custom set combines the data in the 20-150 range, which results in a further decreased performance compared to 30 bp model that excludes the 30-60 bin (or the model using only the 30-60 bin), indicating that the loss of information when removing the 30-60 bin is at least in part compensated by further granularity in the 60-150 bp range. Finally, comparing the 10, 50 and 100 bp models indicates that increasing the granularity may slightly improve the performance of the model although all of the models performed well and none of these schemes reaches the performance of the 30 bp P30_60 model (dashed line).

### Discussion

Tumour-derived DNA has previously been detected in the CSF of patients with glioma and may be helpful for tumour genomic analysis (De Mattos-Arruda et al, 2015; Pentsova et al, 2016; Wang et al, 2015; Pan et al, 2015; Miller et al, 2019; Mouliere et al, 2018b). However difficulties with longitudinal CSF collection in patients alongside the relative variability in tumour fraction detection may hamper clinical implementation and applicability of CSF analysis. There were different observations reported on the level of detection of ctDNA in plasma of glioma patients (Bettegowda et al, 2014; Pan et al, 2019; Mouliere et al, 2018a; Westphal & Lamszus, 2015). No prior studies had, to our knowledge, explored ctDNA analysis in urine samples from glioma patients.

Here, the inventors have shown that ctDNA can be detected, at very low levels, in the urine and plasma of the majority of patients with high grade glioma. The inventors identified size differences between mutant and non-mutant DNA using tumour-guided sequencing in CSF, plasma and urine of glioma patients. They analysed the size distributions of mutant ctDNA by sequencing >435 potentially mutated loci per patient at high depth. This revealed reads that could be unequivocally identified as tumour derived, and allowed a direct comparison of fragmentation features of ctDNA as compared to bulk cfDNA. Whilst a powerful technique, a potential limitation of this method is the fact that capture-based sequencing may be biased by probe capture efficiency and therefore may not accurately reflect ratios between tumour and non-tumour DNA, especially for short fragments <100 bp. Nevertheless, this observation was important as it strongly suggested that ctDNA size shift could be observed in the plasma and the urine of glioma patients. In the case of the former, this agrees with previous data generated using non capture based methods.

The inventors complemented this observation by analysing the genome-wide fragmentation patterns of urine cfDNA in 40 samples from 35 glioma patients using sWGS. They identified cfDNA fragmentation features that could classify urine samples from glioma patients from controls using urine samples, without a priori knowledge of somatic aberrations. The median size of cfDNA fragments in urine from control individuals without glioma (137 bp), patients with other CNS diseases (121 bp) and patients with gliomas (101 bp) was different from previous reports on other cancer types (Cheng et al, 2019; Markus et al, 2021). This could indicate that the cfDNA fragmentation profile could be biased depending on the collection procedure and pre-analytical factors. It is also possible that the shortening of cfDNA in the urine of glioma patients compared to controls is due, at least in part, to differences in patient physiology and that this may directly contribute to the detection of a fragmentation based glioma cfDNA signal in urine. Beyond the tissue of cancer origin, it is likely that urine cfDNA fragmentation might also be influenced by patient physiology (Teo et al, 2019), and pre-analytical parameters (Bosschieter et al, 2018). We attempted to mitigate for these effects by assessing the effect of age on the cfDNA fragmentation of urine samples, by controlling for the duration of pre-operative fasting, by using standardised sample preparation and DNA isolation and also by assessing the effect of tumour size on detectability. A more in depth analysis of how biological variables impact cfDNA fragmentation in urine samples will be needed in order to conclude the extent to which these factors may lead to different fragmentation patterns in different cohorts. Such pre-analytical differences notwithstanding, by using a binary classification the inventors observed that the shorter size ranges (P30-60 and P61-90) of cfDNA fragments in urine samples showed larger differences between cancer cases and controls. These size ranges were similar to the size range enriched in mutant cfDNA in urine as observed using tumour-guided capture panels. With 4 machine learning analyses, they identified and tested ten size features that can be informative for classifying urine samples as being derived either from healthy individuals or from patients with glioma. The LR, RF, SVM and GLMEN models correctly classified samples derived from patients with glioma in most of the cases (median AUC=0.90, median AUC=0.91, median AUC=0.80 and median AUC=0.91, respectively). The GLMEN model correctly identified samples from cancer patients vs samples from controls with a sensitivity of 65% and specificity of 95% in a cohort of 93 urine samples (40 cancer samples and 53 control samples). These results from urine samples from glioma show similar performance to those demonstrated in plasma in the inventors' previous work, which identified 63% of plasma samples from glioma patients with 94% specificity using another RF model based on integration of fragmentation features in plasma cfDNA (Mouliere et al, 2018a). Together with other studies that utilise methylation patterns in plasma (Sabedot et al, 2021; Nassiri et al, 2020), our work suggests that despite a low detection rate of mutations, epigenetic signals (i.e. fragmentation patterns) can be robustly detected in the plasma and also urine of glioma patients.

Thus, the inventors have demonstrated that classification algorithms can utilise information derived from cfDNA fragmentation features to improve the detection of glioma in patients using urine samples. These techniques may therefore provide a method to detect glioma in a truly non-invasive (urine) manner and thus avoiding the morbidity and risk of mortality associated with CSF sampling. These results encourage further confirmation through the analysis of a larger cohort of both glioma patients and control individuals without cancer.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

Best M G, Sol N, Tannous B A, Wesseling P & Wurdinger T (2015) RNA-Seq of Tumor-Educated Platelets Enables Blood-Based Pan-Cancer, Multiclass, and Molecular Pathway Cancer Diagnostics. Cancer Cell 28: 666-676
Bettegowda C, Sausen M, Leary R J, Kinde I, Wang Y, Agrawal N, Bartlett B R, Wang H, Luber B, Alani R M, et al (2014) Detection of circulating tumor DNA in early- and late stage human malignancies. Sci Transl Med 6: 224ra24
Bosschieter J, Bach S, Bijnsdorp I V., Segerink L I, Rurup W F, van Splunter A P, Bahce I, Novianti P W, Kazemier G, van Moorselaar R J A, et al (2018) A protocol for urine collection and storage prior to DNA methylation analysis. PLoS One 13: e0200906
Brennan C W, Verhaak R G W, McKenna A, Campos B, Noushmehr H, Salama S R, Zheng S, Chakravarty D, Sanborn J Z, Berman S H, et al (2013) The somatic genomic landscape of glioblastoma. Cell 155: 462-77
Burnham P, Kim M S, Agbor-Enoh S, Luikart H, Valantine H A, Khush K K & De Vlaminck I (2016) Single-stranded DNA library preparation uncovers the origin and diversity of ultrashort cell-free DNA in plasma. Sci Rep 6: 27859
Cheng T H T, Jiang P, Teoh J Y C, Heung M M S, Tam J C W, Sun X, Lee W S, Ni M, Chan R C K, Ng C F, et al (2019) Noninvasive detection of bladder cancer by shallow-depth genome wide bisulfite sequencing of urinary cell-free DNA for methylation and copy number profiling. Clin Chem 65: 927-936
Du Clos T W, Volzer M A, Hahn F F, Xiao R, Mold C & Searles R P (1999) Chromatin clearance in C57B1/10 mice: Interaction with heparan sulphate proteoglycans and receptors on Kupffer cells. Clin Exp Immunol 117: 403-411
Dudley J C, Schroers-Martin J, Lazzareschi D V., Shi W Y, Chen S B, Esfahani M S, Trivedi D, Chabon J J, Chaudhuri A A, Stehr H, et al (2019) Detection and surveillance of bladder cancer using urine tumor DNA. Cancer Discov 9: 500-509
Engelborghs S, Niemantsverdriet E, Struyfs H, Blennow K, Brouns R, Comabella M, Dujmovic I, van der Flier W, Frölich L, Galimberti D, et al (2017) Consensus guidelines for lumbar puncture in patients with neurological diseases. Alzheimer's Dement Diagnosis, Assess Dis Monit 8: 111-126
Gauthier V J, Tyler L N & Mannik M (1996) Blood clearance kinetics and liver uptake of mononucleosomes in mice. J Immunol 156: 1151-6
Hasbun R, Abrahams J, Jekel J & Quagliarello V J (2001) Computed Tomography of the Head before Lumbar Puncture in Adults with Suspected Meningitis. N Engl J Med 345: 1727-1733
Hentschel A E, Nieuwenhuijzen J A, Bosschieter J, van Splunter A P, Lissenberg-Witte B I, van der Voorn J P, Segerink L I, van Moorselaar R J A & Steenbergen R D M (2020) Comparative Analysis of Urine Fractions for Optimal Bladder Cancer Detection Using DNA Methylation Markers. Cancers (Basel) 12: 859
Husain H, Melnikova V O, Kosco K, Woodward B, More S, Pingle S C, Weihe E, Park B H, Tewari M, Erlander M G, et al (2017) Monitoring Daily Dynamics of Early Tumor Response to Targeted Therapy by Detecting Circulating Tumor DNA in Urine. Clin Cancer Res 23: 4716-4723
Kim J, Lee I H, Cho H J, Park C K, Jung Y S, Kim Y, Nam S H, Kim B S, Johnson M D, Kong D S, et al (2015) Spatiotemporal Evolution of the Primary Glioblastoma Genome. Cancer Cell
Kros J M, Mustafa D M, Dekker L J M, Smitt PAES, Luider T_{M} & Zheng P P (2015) Circulating glioma bi 790 omarkers. Neuro Oncol 17: 343-360 doi: 10.1093/neuonc/nou207
Mair R, Mouliere F, Smith C G, Chandrananda D, Gale D, Marass F, Tsui D W Y, Massie C E, Wright A J, Watts C, et al (2019) Measurement of plasma cell-free mitochondrial tumor DNA improves detection of glioblastoma in patient-derived orthotopic xenograft models. Cancer Res 79: 220-230
Markus H, Zhao J, Contente-Cuomo T, Stephens M D, Raupach E, Odenheimer-Bergman A, Connor S, McDonald B R, Moore B, Hutchins E, et al (2021) Analysis of recurrently protected genomic regions in cell-free DNA found in urine. Sci Transl Med 13
De Mattos-Arruda L, Mayor R, Ng C K Y, Weigelt B, Martínez-Ricarte F, Torrejon D, Oliveira M, Arias A, Raventos C, Tang J, et al (2015) Cerebrospinal fluid-derived circulating tumour DNA better represents the genomic alterations of brain tumours than plasma. Nat Commun 6: 8839
Miller A M, Shah R H, Pentsova E I, Pourmaleki M, Briggs S, Distefano N, Zheng Y, Skakodub A, Mehta S A, Campos C, et al (2019) Tracking tumour evolution in glioma through liquid biopsies of cerebrospinal fluid. Nature 565: 654-658
Moss J, Magenheim J, Neiman D, Zemmour H, Loyfer N, Korach A, Samet Y, Maoz M, Druid H, Arner P, et al (2018) Comprehensive human cell-type methylation atlas reveals origins of circulating cell-free DNA in health and disease. Nat Commun 9: 5068
Mouliere F, Chandrananda D, Piskorz A M, Moore E K, Morris J, Ahlborn L B, Mair R, Goranova T, Marass F, Heider K, et al (2018a) Enhanced detection of circulating tumor DNA by fragment size analysis. Sci Transl Med 10: eaat4921
Mouliere F, Mair R, Chandrananda D, Marass F, Smith C G, Su J, Morris J, Watts C, Brindle K M & Rosenfeld N (2018b) Detection of cell-free DNA fragmentation and copy number alterations in cerebrospinal fluid from glioma patients. EMBO Mol Med 10: e9323
Mouliere F, El Messaoudi S, Pang D, Dritschilo A & Thierry A R (2014) Multi-marker analysis of circulating cell-free DNA toward personalized medicine for colorectal cancer. Mol Oncol 8: 927-941
Mouliere F, Robert B, Peyrotte E, Del Rio M, Ychou M, Molina F, Gongora C & Thierry A R (2011) High fragmentation characterizes tumour-derived circulating DNA. PLoS One 6: e23418
Nassiri F, Chakravarthy A, Feng S, Shen S Y, Nejad R, Zuccato J A, Voisin M R, Patil V, Horbinski C, Aldape K, et al (2020) Detection and discrimination of intracranial tumors using plasma cell-free DNA methylomes. Nat Med 26: 1044-1047
Nørøxe DS, Østrup O, Yde C W, Ahlborn L B, Nielsen F C, Michaelsen S R, Larsen V A, Skjøth-Rasmussen J, Brennum J, Hamerlik P, et al (2019) Cell-free DNA in newly diagnosed patients with glioblastoma—a clinical prospective feasibility study. Oncotarget 10: 4397-4406
Pan C, Diplas B H, Chen X, Wu Y, Xiao X, Jiang L, Geng Y, Xu C, Sun Y, Zhang P, et al (2019) Molecular profiling of tumors of the brainstem by sequencing of CSF-derived circulating tumor DNA. Acta Neuropathol 137: 297-306
Pan W, Gu W, Nagpal S, Gephart M H & Quake S R (2015) Brain tumor mutations detected in cerebral spinal fluid. Clin Chem 61: 514-522
Patel K M, Van Der Vos K E, Smith C G, Mouliere F, Tsui D, Morris J, Chandrananda D, Marass F, Van Den Broek D, Neal D E, et al (2017) Association of Plasma and Urinary Mutant DNA with Clinical Outcomes in Muscle Invasive Bladder Cancer. Sci Rep 7:5554
Pentsova E I, Shah R H, Tang J, Boire A, You D, Briggs S, Omuro A, Lin X, Fleisher M, Grommes C, et al (2016) Evaluating cancer of the central nervous system through next generation sequencing of cerebrospinal fluid. J Clin Oncol 34: 2404-2415
Piccioni D E, Achrol A S, Kiedrowski L A, Banks K C, Boucher N, Barkhoudarian G, Kelly D F, Juarez T, Lanman R B, Raymond V M, et al (2019) Analysis of cell-free circulating tumor DNA in 419 patients with glioblastoma and other primary brain tumors. CNS Oncol 8: CNS34
van der Pol Y & Mouliere F (2019) Toward the Early Detection of Cancer by Decoding the Epigenetic and Environmental Fingerprints of Cell-Free DNA. Cancer Cell 36: 350-368
Sabedot T, Malta T, Snyder J, Nelson K, Wells M, DeCarvalho A, Mukherjee A, Chitale D, Mosella M, Sokolov A, et al (2021) A serum-based DNA methylation assay provides accurate detection of glioma. Neuro Oncol
Seoane J, De Mattos-Arruda L, Rhun E Le, Bardelli A & Weller M (2019) Cerebrospinal fluid cell-free tumour DNA as a liquid biopsy for primary brain tumours and central nervous system metastases. Ann Oncol 30: 211-218 doi: 10.1093/annonc/mdy544
Shen S Y, Singhania R, Fehringer G, Chakravarthy A, Roehrl M H A, Chadwick D, Zuzarte P C, Borgida A, Wang T T, Li T, et al (2018) Sensitive tumour detection and classification using plasma cell-free DNA methylomes. Nature 563: 579-583 doi:10.1038/s41586-018-0703-0
Smith C G, Moser T, Mouliere F, Field-Rayner J, Eldridge M, Riediger A L, Chandrananda D, Heider K, Wan J C M, Warren A Y, et al (2020) Comprehensive characterization of cell free tumor DNA in plasma and urine of patients with renal tumors. Genome Med 12: 23
Teo Y V, Capri M, Morsiani C, Pizza G, Faria A M C, Franceschi C & Neretti N (2019) Cell-free DNA as a biomarker of aging. Aging Cell 18: e12890
Underhill H R, Kitzman J O, Hellwig S, Welker N C, Daza R, Baker D N, Gligorich K M, Rostomily R C, Bronner M P & Shendure J (2016) Fragment Length of Circulating Tumor DNA. PLoS Genet 12: e1006162
Wan J C M, Heider K, Gale D, Murphy S, Fisher E, Mouliere F, Ruiz-Valdepenas A, Santonja A, Morris J, Chandrananda D, et al (2020) ctDNA monitoring using patient-specific sequencing and integration of variant reads. Sci Transl Med 12
Wang Y, Springer S, Zhang M, McMahon K W, Kinde I, Dobbyn L, Ptak J, Brem H, Chaichana K, Gallia G L, et al (2015) Detection of tumor-derived DNA in cerebrospinal fluid of patients with primary tumors of the brain and spinal cord. Proc Natl Acad Sci US A 112: 9704-9709
Wesseling P & Capper D (2018) WHO 2016 Classification of gliomas. Neuropathol Appl Neurobiol 44: 139-150
Westphal M & Lamszus K (2015) Circulating biomarkers for gliomas. Nat Rev Neurol 11:556-566 doi: 10.1038/nrneurol.2015.171
Zill O A, Banks K C, Fairclough S R, Mortimer S A, Vowles J V., Mokhtari R, Gandara D R, Mack P C, Odegaard J I, Nagy R J, et al (2018) The landscape of actionable genomic alterations in cell-free circulating tumor DNA from 21, 807 advanced cancer patients. Clin Cancer Res 24: 3528-3538

1. A method for analysing a urine sample from a subject, the method comprising:
providing the value of one or more cell-free DNA fragment size metrics for said sample;
determining whether the sample has a high or low likelihood of being from a brain cancer patient by providing said values of said cell-free DNA fragment size metrics as input to a machine learning model trained to classify sample data into one of at least two classes, the at least two classes comprising a first class having a high likelihood of being from a brain cancer patient and a second class having a low likelihood of being from a brain cancer patient, wherein the one or more cell-free DNA fragment size metrics comprise at least one metric representing the proportion of fragments in a size range that does not extend above 100 bp and that is between 10 and 100 bp wide.

2. The method of any preceding claim, wherein the one or more cell-free DNA fragment size metrics comprise a plurality of metrics representing the proportion of fragments in respective size ranges, optionally wherein the respective size ranges are substantially non-overlapping and/or wherein the one or more cell-free DNA fragment size metrics comprises a metric representing the amplitude of oscillations in fragment size density with approximately 10 bp periodicity in a particular size range, optionally wherein the particular size range is between approximately 50 bp and approximately 140 bp.

3. The method of any preceding claim, wherein the one or more cell-free DNA fragment size metrics comprise a plurality of metrics representing the proportion of fragments in respective size ranges that are each between 0 and 300 bp, optionally wherein each of the respective size ranges is between 10 and 100 bp wide.

4. The method of any preceding claim, wherein the one or more cell-free DNA fragment size metrics comprise a plurality of metrics representing the proportion of fragments in respective substantially non-overlapping size ranges between 0 and 150 bp, optionally wherein the one or more cell-free DNA fragment size metrics comprise at least 2 or at least 3 metrics representing the proportion of fragments in respective substantially non-overlapping size ranges between 0 and 150 bp.

5. The method of any preceding claim, wherein the size range or each of the respective size ranges is between 20 and 100 bp wide, between 20 and 80 bp wide, between 20 and 50 bp wide, at least 10 bp wide, at least 20 bp wide, at least 30 bp wide, at most 100 bp wide, at most 90 bp wide, at most 80 bp wide, at most 70 bp wide, at most 60 bp wide, at most 50 bp wide, about 20 bp wide, about 30 bp wide, about 40 bp wide or about 50 bp wide.

6. The method of any preceding claim, wherein the one or more cell-free DNA fragment size metrics comprise one or more metrics representing the proportion of fragments in the 30-90 bp range and/or one or more metrics representing the proportion of fragments in the 90-150 bp range,
optionally wherein the one or more metric representing the proportion of fragments in the 30-90 bp range comprises a metric representing the proportion of fragments in the 30-60 bp range and/or a metric representing the proportion of fragments in the 60-90 bp range, and/or wherein the one or more metric representing the proportion of fragments in the 90-150 bp range comprises a metric representing the proportion of fragments in the 90-120 bp range and/or a metric representing the proportion of fragments in the 120-150 bp range.

7. The method of any preceding claim, wherein the one or more cell-free DNA fragment size metrics comprise a metric representing the proportion of fragments in a plurality of ranges selected from the following ranges: 30-60 bp, 60-90 bp, 90-120 bp, 120-150, 150-180, 180-210, 240-270 and 270-300, optionally wherein the cell-free DNA fragment size metrics further comprise a metric representing the amplitude of oscillations in fragment size density with 10 bp periodicity in a particular size range and/or a metric representing the proportion of fragments in each of the following ranges: 30-60 bp, 60-90 bp, 90-120 bp, 120-150, 150-180, 180-210, 240-270 and 270-300.

8. The method of any preceding claim, wherein providing the value of one or more cell-free DNA fragment size metrics for said sample comprises:
providing data representing fragment sizes of cell-free DNA fragments obtained from said sample; and
determining the value of the one or more cell-free DNA fragment size metrics from the data representing fragment sizes of cell-free DNA fragments obtained from said sample, optionally wherein the step of providing data representing fragment sizes of cell-free DNA fragments obtained from said sample comprises sequencing DNA from said sample and/or obtaining a urine sample from said subject and/or processing a urine sample from said subject or a sample of DNA derived therefrom.

9. The method of any preceding claim, wherein the value of one or more cell-free DNA fragment size metrics for said sample is/are derived from sequence data, optionally wherein the sequence data is whole genome sequencing (WGS) data, paired-end sequencing data, hybrid-capture sequencing and/or shallow whole genome sequencing (sWGS) data.

10. The method of any preceding claim, wherein the machine learning model has been trained using training data comprising the values of cfDNA size metrics for a plurality of urine samples from subjects with brain cancer and for a plurality of urine samples from subjects that do not have brain cancer, optionally wherein the subjects that do not have brain cancer comprise healthy subjects and subjects with non-malignant central nervous system diseases.

11. The method of any preceding claim, wherein the machine learning model is a random forest model, a logistic regression model, a support vector machine, or a generalised linear model, optionally a regularized generalised linear model.

12. The method of any preceding claim, wherein the urine sample is from a subject having or suspected of having a brain cancer, and/or wherein the brain cancer is a glioma, a meningioma, a pituitary adenoma, a glioblastoma, a medulloblastoma, an oligodendroglioma, a brain metastasis, optionally wherein the brain cancer is a glioma, and/or wherein the subject is a human.

13. The method of any preceding claim, wherein the method is for detecting the presence of, growth of, prognosis of, regression of, treatment response of, residual disease or recurrence of a brain cancer in a subject from which the sample has been obtained.

14. The method of any preceding claim, wherein the urine sample has been obtained prior to the subject having undergone treatment with a cancer therapy, wherein the urine sample has been obtained subsequent to the subject having undergone treatment with a cancer therapy, and/or wherein the method is carried out on a sample obtained prior to a cancer treatment of the subject and on a sample obtained following the cancer treatment of the subject.

15. The method of any preceding claim, wherein the urine sample has been processed within 12 hours, within 4 hours, within 2 hours or within an hour of collection, optionally wherein the processing comprises refrigeration, freezing, centrifugation, and/or mixing with one or more preserving compounds such as EDTA.

16. A method of diagnosing a subject suspected of having a brain cancer as likely to have brain cancer, the method comprising:
analysing one or more urine samples from the subject using the method of any preceding claim to determine whether the one or more samples have a high or low likelihood of being from a brain cancer patient; and
diagnosing the subject as likely to have a brain cancer if one or more of the one or more urine samples are determined to have a high likelihood of being from a brain cancer patient.

17. A method of selecting a subject suspected of having a brain cancer for treatment with a cancer therapy, the method comprising characterising a urine sample obtained from the subject as having a high or low likelihood of being from a cancer patient using the method of any of claims 1 to 15, and selecting the subject for treatment with the cancer therapy if the sample is characterised as

18. A method of selecting a subject suspected of having a brain cancer for further diagnostic test, optionally wherein the further diagnostic test is an invasive diagnostic test or an imaging-based test, the method comprising characterising a urine sample obtained from the subject as having a high or low likelihood of being from a cancer patient using the method of any of claims 1 to 15, and selecting the subject for further diagnostic test if the sample is characterised as having a high likelihood of being from a brain cancer patient.

19. A method of detecting recurrence and/or residual disease of a brain cancer in a subject, the method comprising characterising a urine sample obtained from the subject as having a high or low likelihood of being from a cancer patient using the method of any of claims 1 to 15, and determining that recurrence is likely to have occurred and/or residual disease is likely to be present if the sample is characterised as having a high likelihood of being from a brain cancer patient.

20. The method of any preceding claim, wherein the subject has been previously treated for brain cancer, and/or wherein the method is repeated using urine samples that have been obtained from the subject at a plurality of subsequent times to monitor the presence or absence of recurrence of a brain cancer in the subject.

21. The method of any preceding claim, further comprising outputting a result of the method, optionally wherein the result is selected from a classification of a sample in the high/low likelihood class, a probabilistic score indicating the likelihood of the sample being from a brain cancer patient, or information derived therefrom such as a prognosis, therapeutic or diagnosis indication.

22. A method for providing a tool for analysing a urine sample, the method comprising:
providing the value of one or more cell-free DNA fragment size metrics for a plurality of training urine samples associated with known brain cancer status, wherein the one or more cell-free DNA fragment size metrics comprise at least one metric representing the proportion of fragments in a size range that does not extend above 100 bp and that is between 10 and 100 bp wide; and
training a machine learning model to classify sample data into one of at least two classes, the at least two classes comprising a first class having a high likelihood of being from a brain cancer patient and a second class having a low likelihood of being from a brain cancer patient.

23. A system a system comprising:
a processor; and
a computer readable medium comprising instructions that, when executed by the processor, cause the processor to perform the steps of the method of any of claims 1 to 22.

24. A non-transitory computer readable medium or media comprising instructions that, when executed by at least one processor, cause the at least one processor to perform the method of any of claims 1 to 22.
